(19) United States
(12) Patent Application Publication
(10) Pub. No.: US 2010/0068142 A1
(43) Pub. Date: Mar. 18, 2010

(54) MELANOMA REPOPULATING CELLS

(76) Inventors: Craig Gedye, Heidelberg (AU); Ian Davis, Warrandyte (AU); Jonathan S. Cebon, Clifton Hill (AU)

Correspondence Address: WOLF GREENFIELD & SACKS, P.C., 600 ATLANTIC AVENUE, BOSTON, MA, 02210-2206, US

(21) Appl. No.: 12/311,508

(22) PCT Filed: Oct. 3, 2007
(86) PCT No.: PCT/US2007/021239

§ 371 Date: Dec. 4, 2009

Related U.S. Application Data
(60) Provisional application No. 60/848,913, filed on Oct. 3, 2006.

(51) Int. Cl. A61K 49/00; C12N 5/00; A61K 39/395; G01N 33/574

(52) U.S. Cl. 424/9.1; 435/325; 424/138.1; 435/375; 435/7.23

(57) ABSTRACT

The invention relates to the identification of melanoma repopulating cells, characterization of these cells, and diagnostic and therapeutic methods based on an understanding of the properties of these cells.

### RELATED APPLICATION

This application claims the benefit under 35 U.S.C. §119(e) of U.S. provisional patent application Ser. No. 60/848,913, filed Oct. 3, 2006, the content of which is incorporated herein by reference in its entirety.

### FIELD OF THE INVENTION

The invention relates to the identification of melanoma repopulating cells, characterization of these cells, and diagnostic and therapeutic methods based on an understanding of the properties of these cells.

### BACKGROUND OF THE INVENTION

There is increasing recognition that, like non-malignant tissues, cancers are populated from a clonogenic subset of cells. These have been come to be known as cancer stem cells. While a stem cell is largely accepted in acute myeloid leukemia¹, there is some criticism of a similar hierarchical structure in solid tumours². The cancer stem cell hypothesis is consistent with many clinical and biological observations of cancer behaviour³ and evidence for the hypothesis has been demonstrated in leukaemia¹, breast carcinoma⁴ and brain neoplasms⁵. It has been proposed that the ability of metastatic cancer to migrate reflects the migrating nature of cancer stem cells⁶ and repopulating cells have been identified as an important cause of treatment failure⁷.

The repopulation of non-malignant tissues is a tightly controlled process in which undifferentiated stem cells self-renew and also provide progenitor cells which progressively yield differentiated cells with reduced capacity to proliferate. Consequently a common stem cell can give rise to cells which differentiate along multiple distinct lineages. The capacity to both self-renew and populate tissues with multiple lineages of cells provides a functional definition. In contrast cancer cells have generally lost the tight regulatory controls which govern differentiation and its link with proliferation. Cancer cells with repopulating potential may therefore fail a strict definition for ‘stemness’, nonetheless are biologically critical targets for cancer therapy since effective therapy necessitates the eradication of these cells.

It has been hypothesized that melanoma is propagated by an intrinsic stem-cell-like population, with the first evidence being that only a fraction of cells were clonogenic in an in vitro agar culture⁸. More recently Fang et al. have reported a CD20-positive subpopulation of melanoma cell lines that exhibited stem-cell like behaviour⁹. Using Hoechst dye exclusion Grichnik et al. demonstrated a subpopulation in melanoma cell lines that had stem cell-like properties¹⁰ and Frank et al. have recently reported that a subpopulation of melanoma cells co-express CD133 and the drug transporter ABCB5 that are more resistant to chemotherapy, and have enhanced clonogenicity¹¹.

A need therefore exists for better identification of cells that give rise to and/or propagate melanoma to facilitate improved detection and therapy of melanoma.

### SUMMARY OF THE INVENTION

Cancer stem cells have been identified in several malignancies and have been proposed to exist in melanoma. We report a subpopulation of clonogenic melanoma cells, termed “melanoma repopulating cells” (MRCs), that can be identified and enriched in some (−20%) melanoma cell lines by high CD133 expression. CD133hi cells can also be identified ex vivo in resected human melanoma specimens. The CD133hi cells identified in these cell lines have enhanced clonogenicity and the ability to self renew in vitro, but have similar proliferative rates compared with CD133neg cells. As used herein, “clonogenic” means that the cells are clones of one another, as is shown by the ability to produce colonies in standard assays such as soft agar assays.

In some lines CD133hi melanoma cells express high levels of cancer-testis antigens (CTAg), however expression of differentiation antigens is similar to CD133neg cells. This high expression of cancer-testis antigens in clonogenic melanoma cells allows them to be targeted for killing in vitro by cancer-testis antigen-specific CD8+ T-lymphocytes. We have demonstrated that NY-ESO-1 specific cytotoxic T-lymphocytes recognize melanoma cells in vitro, and can target and eliminate the clonogenic stem-like melanoma repopulating cells. Cancer-testis antigens such as NY-ESO-1 may be relevant to the biology of MRCs and are immunological targets for adjuvant melanoma vaccine design.

According to one aspect of the invention, an isolated cell population is provided that includes melanoma repopulating cells (MRCs). The MRCs express CD133 and at least one cancer-testis antigen (CTAg), and are clonogenic. In some embodiments, the CD133 is CD133-1 or a splice variant thereof, and preferably the splice variant is CD133-2. In other embodiments, the MRCs have an increased expression of one or more cancer-testis antigens (CTAgs), in amount or type of CTAgs, relative to melanoma cells that are not MRCs. In preferred embodiments, the MRCs express one or more of NY-ESO-1, MAGEA3, MAGEA4, MAGEA1 and CT7. In some embodiments, the expression of NY-ESO-1, MAGEA3, MAGEA4, MAGEA1, and/or CT7 is higher in the MRCs than in melanoma cells (of the same origin) that are not MRCs; preferably the melanoma cells that are not MRCs do not express CD133. In some embodiments, the expression of one or more of NY-ESO-1, MAGEA3, MAGEA4, MAGEA1 and CT7 is determined by immunohistochemistry or RT-PCR.

In other embodiments, the MRCs also express one or more neural lineage stem-cell markers. Preferably the one or more neural lineage stem-cell markers are nestin, PSA-NCAM, α-tubulin type III and/or microtubule-associated protein 2 (MAP2).

In still other embodiments, the MRCs also express one or more markers for stem cells. Preferably, the one or more markers for stem cells are the SSEAs, ABCG2 and/or Sca-1.

In some embodiments, the MRCs are isolated by contacting a population of melanoma cells with an antibody that binds CD133 and the contacted cells are isolated by flow cytometry. Preferably the antibody is detectably labeled. The contacted cells in some embodiments are further contacted with a detectably labeled molecule that binds to the antibody. In other embodiments, the MRCs are isolated by contacting a population of melanoma cells with immunomagnetic particles, such as beads, comprising an antibody that binds to CD133, and isolating the immunomagnetic particles.

The MRCs have certain growth characteristics. In some embodiments, the MRCs form colonies in soft agar and/or proliferate without feeder cells or conditioned medium.

In some embodiments, the MRCs are enriched in the cell population by at least 2-fold relative to an unenriched cell population. Preferably the MRCs are enriched in the cell population by at least 5-fold relative to an unenriched cell population, more preferably the MRCs are enriched in the cell population by at least 10-fold relative to an unenriched cell population, still more preferably the MRCs are enriched in the cell population by at least 20-fold relative to an unenriched cell population, and yet more preferably the MRCs are enriched in the cell population by at least 50-fold relative to an unenriched cell population.

According to another aspect of the invention, methods for treating cancer are provided. The methods include administering to a subject an effective amount of an agent or combination of agents selectively targeted to MRCs of the population of melanoma cells. The agent or combination of agents kills the MRCs or inhibits the proliferation of MRCs. In some embodiments, the population of melanoma cells is a tumor.

In some embodiments, the agent is an antibody or antigen-binding fragment thereof that binds CD133 and/or a CTAg. Preferably the agent is a bispecific or multispecific antibody that binds CD133 and at least one CTAg. In some embodiments, the at least one CTAg is one or more of NY-ESO-1, MAGEA3, MAGEA4, MAGEA1 and CT7.

In other embodiments, the agent is labeled with a cytotoxic agent or a cytostatic agent; preferably the cytotoxic agent is a radionuclide.

In still other embodiments, the agent reduces expression of one or more CTAgs, preferably NY-ESO-1. Preferably the agent comprises one or more small interfering RNA molecules (siRNA) or other nucleic acid molecules that reduce expression of the one or more CTAgs by RNA interference. In some preferred embodiments, the agent comprises one or more double stranded RNA molecules.

In yet other embodiments, the combination of agents comprises an antibody or antigen-binding fragment thereof that binds CD133 and one or more small interfering RNA molecules (siRNA) or other nucleic acid molecules that reduce expression of the one or more CTAgs by RNA interference, preferably NY-ESO-1.

According to another aspect of the invention, methods for killing or inhibiting the proliferation of melanoma repopulating cells (MRCs) are provided. The methods include contacting a population of melanoma cells with an agent or combination of agents selectively targeted to MRCs of the population of melanoma cells, wherein the agent or combination of agents kills the MRCs or inhibits the proliferation of MRCs. In some embodiments, the population of melanoma cells is a tumor.

In some embodiments, the agent is an antibody or antigen-binding fragment thereof that binds CD133 and/or a CTAg. Preferably the agent is a bispecific or multispecific antibody that binds CD133 and at least one CTAg. In some embodiments, the at least one CTAg is one or more of NY-ESO-1, MAGEA3, MAGEA4, MAGEA1 and CT7.

In other embodiments, the agent is labeled with a cytotoxic agent or a cytostatic agent; preferably the cytotoxic agent is a radionuclide.

In still other embodiments, the agent reduces expression of one or more CTAgs, preferably NY-ESO-1. Preferably the agent comprises one or more small interfering RNA molecules (siRNA) or other nucleic acid molecules that reduce expression of the one or more CTAgs by RNA interference. In some preferred embodiments, the agent comprises one or more double stranded RNA molecules.

In yet other embodiments, the combination of agents comprises an antibody or antigen-binding fragment thereof that binds CD133 and one or more small interfering RNA molecules (siRNA) or other nucleic acid molecules that reduce expression of the one or more CTAgs by RNA interference, preferably NY-ESO-1.

According to another aspect of the invention, methods for identifying the presence of melanoma repopulating cells (MRCs) in an animal are provided. The methods include administering to the animal a detectably labeled agent that binds to CD133 and/or a CTAg. In some embodiments, the agent is an antibody or antigen-binding fragment thereof that binds CD133 and/or a CTAg. Preferably the agent is a bispecific or multispecific antibody that binds CD133 and at least one CTAg. In some embodiments, the at least one CTAg is one or more of NY-ESO-1, MAGEA3, MAGEA4, MAGEA1 and CT7. In other embodiments, the detectable label is a contrast agent.

According to another aspect of the invention, methods for identifying the presence of melanoma repopulating cells (MRCs) in a cell sample or tissue sample are provided. The methods include contacting the cell sample or tissue sample with a detectably labeled molecule that binds to CD133 and/or a CTAg. In other embodiments, the agent is an antibody or antigen-binding fragment thereof that binds CD133 and/or a CTAg. Preferably the agent is a bispecific or multispecific antibody that binds CD133 and at least one CTAg. In other embodiments, the at least one CTAg is one or more of NY-ESO-1, MAGEA3, MAGEA4, MAGEA1 and CT7. In other embodiments, the detectable label is a fluorescent molecule. In still other embodiments, the cell sample or tissue sample is a melanoma cell sample or tissue sample.

According to another aspect of the invention, methods for isolating melanoma repopulating cells (MRCs) are provided. The methods include contacting a population of melanoma cells with one or more labeled antibodies that bind to CD133 and/or a cancer-testis antigen (CTAg) and isolating the MRCs based on the binding of the labeled antibodies to the MRCs. In other embodiments, the population of melanoma cells is obtained from a tumor sample or a cell line.

In other embodiments, the labeled antibodies are labeled with fluorescent molecules. In such embodiments, the step of isolating preferably is performed by fluorescence-activated cell sorting or magnetic activated cell sorting.

In other embodiments, the step of contacting the melanoma cells is carried out by first contacting the cells with labeled antibodies that bind to CD133 followed by contacting the cells with labeled antibodies that bind to a CTAg. In still other embodiments, the step of contacting the melanoma cells is carried out by contacting the cells with labeled antibodies that bind to a CTAg first followed by contacting the cells with labeled antibodies that bind to CD133. In alternative embodiments, the step of contacting the melanoma cells is carried out by simultaneously contacting the cells with labeled antibodies that bind to CD133 and with labeled antibodies that bind to a CTAg.

The use of the foregoing compositions in the preparation of medicaments for treatment of disease, particularly cancer, also is provided in accordance with the invention.

These and other aspects of the invention, as well as various embodiments thereof, will become more apparent in reference to the drawings and detailed description of the invention.

### BRIEF DESCRIPTION OF THE DRAWINGS

FIG. 1: (FIG. 1a) A graph plotting survival since the last evidence of melanoma prior to study entry (Davis, Chen et al, PNAS, 2004). In this randomized phase II trial, patients at high risk of relapse from melanoma (resected stage III/IV) who received NY-ESO-1/ISCOMATRIX® vaccine appeared to have improved survival compared to patients who received placebo. (FIG. 1b) A photograph of a representative melanoma specimen from one of the vaccinated trial patients, showing only a minority of melanoma cells express NY-ESO-1. More than half of the patients in the active vaccine group had tumors in which 50% or less of the tumor cells were positive for NY-ESO-1 by IHC.

FIG. 2: Melanoma and melanoma cell lines contain CD133⁺ cells. Photographs of melanoma and melanoma cell lines containing CD133⁺ cells. (FIG. 2a) LM-MeI-34 and LM-Mel-42 melanoma frozen section IHC, (FIG. 2b) cell line IHC.

FIG. 3: Dot plots showing cell line flow cytometry of early passage unsorted cell line (FACS, FIG. 3a), and after magnetic bead cell enrichment (post MACS, FIG. 3b). CD133 expression is variable in the tumours surveyed but mostly represented a minority of cells (boxed).

FIG. 4: Dot plots showing flow cytometry of early and late passage melanoma cell lines. 13 of 32 cell lines and 9 of 23 frozen tumors examined contained CD133⁺ cells (boxed).

FIG. 5: CD133⁺ cells are clonogenic and have stem-like properties compared to CD133″g cells. Photographs of LM-MeI-34 separated by MACS to CD133⁺ (95%, CD133^{hi}) and CD133^{neg}(0.3%) cells in soft agar (FIG. 5a). A plot depicting clonogenic frequency: CD133⁺ LM-MeI-34 cells showing differential clonogenicity compared to control (FIG. 5b). A graph depicting CFU frequency by limiting dilution (FIG. 5c). CD133^{hi} are highly clonogenic compared to CD133^{neg} and control cells.

FIG. 6: Plot depicting clonogenic frequency: CD133 is a marker for clonogenicity in other cell lines (FIG. 6a, LM-MeI-14, and FIG. 6b, LAR42). (FIG. 6c) A graph depicting proliferation measured by relative absorbance: The rate of proliferation of CD133^{hi} cells as measured by formazan production is no different to control or CD133^{neg} cells.

FIG. 7: Graphs depicting flow cytometry. When CD133 separated cells are returned to culture (FIG. 7a, left panel), and reanalyzed by flow cytometry several weeks later, CD133⁺ cells have given rise to CD133^{neg} cells (FIG. 7a, right panel, intermediate profile, containing both CD133⁺ and CD133^{neg} cells), but CD133^{neg} cells have been unable to form CD133⁺ cells (FIG. 7b, compare left and right panel), showing a hierarchical relationship (also in (FIG. 7d, CD133^{hi}) for LAR24 and SK-MeI-28). (FIG. 7c) Photographs depicting singly sorted CD133⁺ melanoma cells showing ongoing proliferation from a single CD133⁺ cell, and the ability to be serially sorted (left panel) versus limited and complex growth from a single CD133^{neg} cell (right panel).

FIG. 8: Photographs showing immunohistochemistry of CD133hi melanoma stem-like cells expressing cancer/testis antigens. LAR34 melanoma cells were MACS separated into CD133hi (top of panel) and CD133neg (bottom). The panels show immunohistochemistry of cell blocks (200×), as follows: (FIG. 8a) CD133; (FIG. 8b) NY-ESO-1; (FIG. 8c) polyMAGE/MAGEA4; (FIG. 8d) MAGEC1; (FIG. 8e) MAGEA1; (FIG. 8f) Melan-A.

FIG. 9: Bar graphs depicting the result of cell counts from randomly selected high-power fields of CD133⁺ (CD133^{hi}) and CD133^{neg} LM-MeI-34 cell blocks (mean±SD) for noted antigens.

FIG. 10: Bar graphs and plots depicting relative expression measured by real-time PCR. (FIG. 10a) LM-MeI-34 CD133^{hi} cells express NY-ESO-1, PRAME and MAGEA10 relative to CD133^{neg} cells. Other cancer/testis and differentiation antigens tested to date were not differentially expressed. (FIG. 10b) LAR24 and SK-Mel-28 CD133^{hi} melanoma cell lines demonstrate high expression of NY-ESO-1 compared to CD133^{neg} and control cells. The legend is the same as in (FIG. 10a). (FIG. 10c) CD133 expression from an initially CD133⁺ enriched population decays over time toward unsorted expression levels, and this decay is paralleled by a decay in CTAg expression. CD133^{neg} cells showed a gradual decline over time in both CD133 and NY-ESO-1 expression. (Squares): Enriched-NY-ESO-1, (Triangles): Enriched-CD133, (Circles): Depleted-CD133, (X): Depleted-NY-ESO-1.

FIG. 11: CD133⁺ melanoma stem-like cells are eliminated by NY-ESO-1 CTL but not by Melan-A specific CTL or chemotherapy. (FIG. 11a) Photographs depicting IHC of unsorted LM-MeI-34 melanoma cell line showing a minority of cells stain positive for NY-ESO-1 (top) compared to Melan-A (bottom; 100×). (FIG. 11b) Plot depicting a clonogenic cell cytotoxicity assay: Unsorted LAR34 melanoma cells (HLA-A2 and HLA-Cw3 positive) were co-incubated with antigen-specific cytotoxic T-lymphocytes (CTL) prior to performing a CFU assay in soft agar. Clonogenicity was eliminated by NY-ESO-192-100 (LAMPFATPM; SEQ ID NO:103) specific CTL, but not by Melan-A26-35 (A27Lsub: ELAGIGILTV; SEQ ID NO:104) specific CTL or irrelevant control EBV BMLF1280-288 (C282Asub: GLATLVAML; SEQ ID NO:105) specific CTL.

FIG. 12: (FIG. 12a) Graph depicting cytotoxicity as measured by chromium release as percent cytotoxicity. LM-MeI-34 target cells can be lysed when co-incubated with increasing ratios of Melan-A effectors, whereas increasing ratios of NY-ESO-1 and EBV specific CTL do not increase lysis. Target melanoma cells loaded with cognate ESO1b peptide can be lysed by NY-ESO-1 effectors. (open squares): Melan-A, (open circles): NY-ESO-1+ peptide, (closed triangles): NY-ESO-1, (closed diamond): EBV. (FIG. 12b) Bar graph depicting flow cytometry as percent viable cells expressing CD133 of LM-MeI-34 melanoma cells after co-incubation with CTLs. While Melan-A CTL may kill many more tumor cells, NY-ESO-1 specific CTL specifically target and eliminate CD133^{k} melanoma stem-like cells which express NY-ESO-1.

FIG. 13: Dot plots depicting intracellular cytokine staining: LAR34 melanoma cells triggered interferon-γ production in Melan-A specific CTL, but little response in NY-ESO-1 or EBV BMLF1 specific CTL (circled).

### DETAILED DESCRIPTION OF THE INVENTION

Given the shared phylogeny of neural cells and melanocytes we hypothesised that CD133, which has been established as a stem cell marker in other systems including neuroepithelium, haemopoietic and endothelial progenitors^{5,12} may mark a stem-cell population in melanoma. CD133 also is known as AC133 and prominin.

Our preliminary studies have confirmed the presence of a subpopulation of cells in melanoma cell lines which have the capacity for clonogenicity and self-renewal and are defined by the presence of CD133 and preferably at least one cancer-testis antigen (CTAg). These cells are referred to herein as Melanoma Repopulating Cells (MRCs). In a number of melanoma cell lines studied, MRCs represent a small subset (1-3%) of total cells. It has been possible to prepare highly enriched populations of these cells and we have characterized them in preliminary experiments. Although they lack the capacity to differentiate into multiple lineages, all clonogenic potential appears to reside in this population. This is not the case for CD13310 cells derived from the same cell lines.

Cancer-testis antigens (CTAgs) are of particular interest in targeting (particularly immune targeting) of cancer because of their favorable tissue distribution and immune characteristics. Although the biological function of CTAgs is still poorly understood, there is emerging evidence that they play a role in the transcriptional regulation of germ cells¹³. This putative role in germ cell biology, linked with the observation that they are frequently re-expressed in cancer provided the rationale to investigate CTAg expression in cancer stem cells. Using a series of melanoma cell lines which were sorted into highly enriched CD133hi and CD133lo cell fractions, MRC were distinguishable from CD13310 cells on the basis of their highly-enriched pattern of CTAg expression. This makes MRC uniquely suitable for immune targeting.

Furthermore, emerging insights into CTAg function also raises the possibility that the inhibition or down-regulation of these molecules may have therapeutic value for the inhibition of MRC proliferation.

Over the last 15 years a large variety of cancer antigens have been identified and clinical trials are currently underway to assess their value as targets for cancer immunotherapy. The cancer-testis antigens¹⁴ have been particularly attractive because their expression is limited to cancer and virtually no non-malignant cells apart from germ cells and trophoblast¹³. Among these, NY-ESO-1 is of particular interest due to the exceptional immunogenicity of this CTAg coupled with its widespread distribution among many cancer types. Expression of NY-ESO-1 protein has been demonstrated in multiple cancer types including melanoma, soft tissue sarcomas, non-small cell lung cancer and cancers of the head & neck, bladder, lung and breast. Normal tissue distribution is limited to spermatogonia, oogonia and trophoblast^{13,15}. These properties makes NY-ESO-1 a very good vaccine candidate, with the potential to be used in vaccines against many types of malignancies. The NY-ESO-1 gene is located on chromosome Xq28¹⁶. Up to 10% of the genes on chromosome X are CTAg genes. Expression of these genes seems to be related to demethylation of the promoter regions since experimental demethylation leads to upregulation of CTAg expression¹⁷⁻²⁶. The NY-ESO-1 gene encodes a protein of 180 amino acids with Mr 18 Kd¹⁶, which is expressed primarily in the cytoplasm although nuclear expression can be seen in some spermatogonia¹⁵. The function of NY-ESO-1 remains unknown. No NY-ESO-1 gene knockout or transgenic studies have been reported in the literature and there is no known rodent homologue of NY-ESO-1. Few of the X-linked CTAg have an ascribed function; for the few that do, the function is mainly related to RNA transcription or translation, or transfer of mRNA to the cytoplasm¹³. Other clues to the function of CTAg might be inferred from observations of patterns of expression in normal tissues in the embryo and postnatal life and in cancer. Expression of CTAg in the testis is restricted to spermatogonia and primary spermatocytes and is rapidly lost during spermatid differentiation^{15,27,28}, suggesting a possible role in germ cell self-renewal or differentiation. Therefore, expression of CTAg by cancers might reflect acquisition or recapitulation of certain properties of germ cells or stem cells that cancers find useful, such as immortality, self renewal, migratory ability and capacity to invade^{15,27,28}.

A placebo-controlled clinical trial was performed to evaluate the safety and immunogenicity of NY-ESO-1 protein formulated in IMX (ESO/IMX)³¹. Forty-six evaluable patients with fully resected NY-ESO-1-positive tumors received three doses of vaccine intramuscularly at monthly intervals. The vaccine was well tolerated. Observed responses included high-titer antibody responses, strong delayed-type hypersensitivity reactions, and circulating CD8(+) and CD4(+) T cells specific for a broad range of NY-ESO-1 epitopes, including known and previously unknown epitopes.

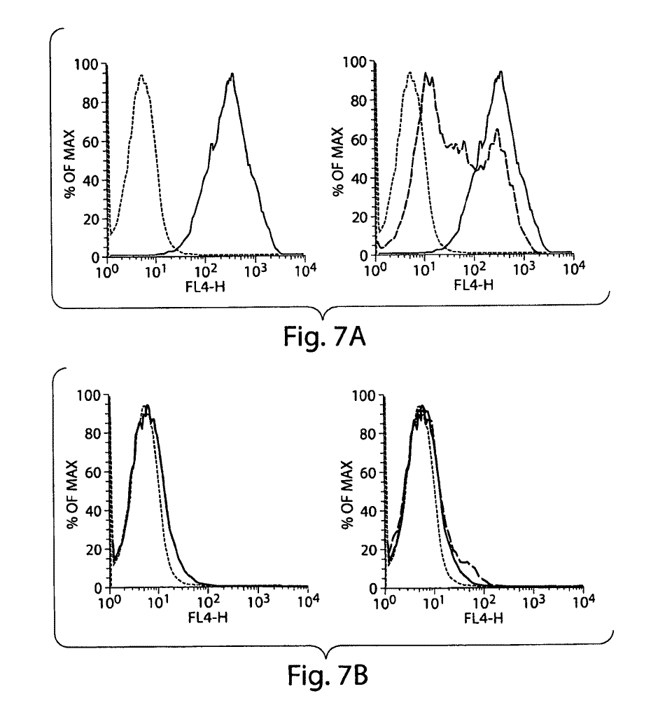

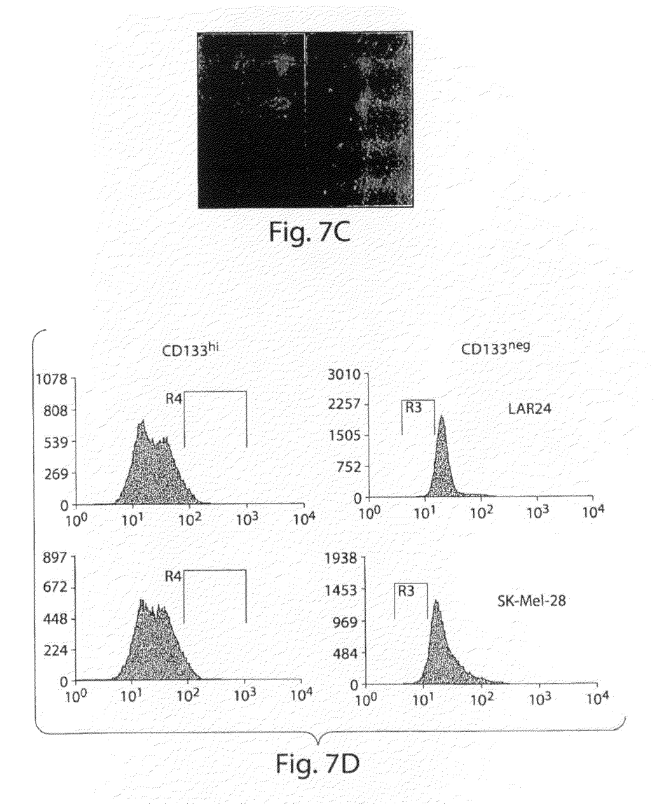

Although this study was not designed to assess clinical endpoints, the impression emerged that most melanoma patients who relapsed were in the groups that were characterized by weaker immune responses, i.e., those who received either placebo or protein alone without adjuvant. Forty-two melanoma patients completed vaccination. With a median exceeding 1000 days, seven or eight placebo patients and only four of 19 who received ESO/IMX have relapsed (FIG. 7a). The following covariates were analyzed retrospectively in case imbalance between groups had affected this result: pathological stage at study entry; primary lesion thickness; age; sex; time since diagnosis; estimated risk of relapse at study entry; number of recurrences before entry; and time since last resection. None were found to be significant³¹.

Since survival was not a protocol-defined objective of this trial these results have been treated with extreme caution, and a confirmatory trial is currently underway. Nonetheless, placebo patients were assigned randomly to treatment concurrently with vaccinated patients and pending the results of the confirmatory trial it cannot be discounted that these results are real. If so, there is a conundrum which needs to be explained, that is, the vaccine appears to have modified survival in recipients many of whom had tumours in which only a minority of cells expressed the NY-ESO-1 antigen. This led us to hypothesize that in those tumours, targeting the minority of NY-ESO-1 Ag+ cells could have had impact if those same cells were also MRCs. FIG. 7b shows that only a minority of melanoma cells express NY-ESO-1 in a representative melanoma specimen from one of the vaccinated trial patients.

The invention provides for the identification, isolation, enrichment, and culture of melanoma repopulating cells that are clonogenic (MRCs). MRCs are identified or selected through the binding of antigens, e.g., as found on the surfaces of MRCs, to reagents that specifically bind such cell surface antigens.

One of these antigens is CD133, which is also known as AC133 and prominin. The AC133 monoclonal antibody is exemplary of antibody embodiments of reagents that recognize CD133 molecules. CD133/AC133/prominin is a membrane protein expressed in various epithelial cells (Weigmann et al., Proc Natl Acad Sci USA. 94(23):12425-12430, 1997; Corbeil et al., J Cell Sci. 112 (Pt 7):1023-1033, 1999; Corbeil et al., Blood 91(7):2625-2626,1998; Miriglia et al., Blood 91(11):4390-4391, 1998).

Various antibodies that bind AC133 (CD133) are described in U.S. Pat. No. 5,843,333, which is incorporated herein by reference. The AC133 antigen is a 5-transmembrane cell surface antigen with a molecular weight of 117 kDa. Expression of this antigen is highly tissue specific, and has been detected on a subset of hematopoietic progenitor cells derived from human bone marrow, fetal bone marrow and liver, cord blood, and adult peripheral blood. These AC133 antibodies are capable of immunoselection for the subset of human cells of interest in this invention. Preferred AC133 monoclonal antibodies can be obtained commercially from Miltenyi Biotec Inc. (Auburn Calif.), including AC133/1-PE antibody (Cat #808-01) and AC133/2-PE antibody (Cat #809-01). For MACS separation, a 50:50 mixture of the monoclonal antibodies is preferred. The high tissue specificity of AC133 expression is particularly advantageous during enrichment for highly purified MRC populations.

Buck, et al. (U.S. Pat. No. 7,037,719) described the isolation of central nervous system (CNS) neural stem cells that express AC133, which can initiate neurospheres (NS—IC) and progenitor cells. The presence of AC133 on cells also has been used to identify, isolate and enrich pancreatic stem cells (Uchida et al., published U.S. Patent Application No. 2006/0205072 A1).

Recently, a novel alternatively spliced variant of human AC133, AC133-2, was reported (Yu et al., J Biol Chem 277:20711-20716, 2002). This novel isoform lacks exon 4, which leads to deletion of 9 amino acids from AC133. According to this report, additional alternative splicing isoforms exist within the open reading frame of human AC133. This report further demonstrates that AC133 transcripts can be alternatively spliced, leading to formation of mRNAs with different 5′ untranslated exons. The mouse AC133 homolog prominin similarly has alternative transcripts.

The invention thus provides isolated cell populations that include melanoma repopulating cells (MRCs). The MRCs express CD133 and are clonogenic. Preferably the MRCs express at least one cancer-testis antigen (CTAg). The CD133 expressed by the MRCs can be any of the isoforms of CD133, such as CD133-1 or a splice variant of CD133. A preferred splice variant is CD133-2.

Cancer-testis antigen expression can be increased in type and/or in amount relative to melanoma cells that are not MRCs. By “increased type” and the like is meant that different and/or more CTAgs are expressed in MRCs relative to non-MRC melanoma cells. By “increased amount” and the like is meant that higher amounts of CTAg are expressed as measured by amounts of RNA transcripts or protein.

Many CTAg are known in the art, see Scanlan et al. Cancer Immun. 2004; 4:1 and Simpson et al., Nat Rev Cancer. 2005; 5:615, which are incorporated by reference herein for information on CTAg. Preferably the MRCs express one or more of NY-ESO-1, MAGEA3, MAGEA4, MAGEA1 and CT7. Most preferably the CTAg is NY-ESO-1.

The expression of CD133 and/or one or more CTAgs, such as NY-ESO-1, MAGEA3, MAGEA4, MAGEA1 and CT7, can be determined by standard methods known in the art. These methods include the immunohistochemistry and RT-PCR methods described below, as well as quantitation of expression using fluorescence activated cell sorting and other methods that allow for discriminating between cell types in a population without significantly affecting the ability of the cells to survive the quantitation methods.

In some cases, the MRCs also express one or more additional stem cell markers, such as neural lineage stem cell markers. Examples of neural lineage stem cell markers include nestin, PSA-NCAM, α-tubulin type III and/or microtubule-associated protein 2 (MAP2). Additional stem cell markers that can be used to identify or isolate MRCs include such markers as Stage Specific Embryonic Antigens (SSEAs), ABCG2 and/or Sca-1.

Additional markers can be used, as needed, to further purify or enrich MRCs. Preferably such markers are cell surface markers, such as ABCB5, CD166, integrin integrin α2β1, SOX10, p75NTR, nestin, BCRP1/ABCG2, CD44, CXCR4/CD184, SSEA-1/CD15, and/or CD24.

Additional markers that are not found on MRCs, but are found on melanoma cells that are not MRCs, also can be used in enrichment and purification methods, e.g., by removing non-MRCs from a population of melanoma cells using various standard methods such as fluorescence activated cell sorting, magnetic activated cell sorting (MACS), killing cells recognized by non-MRC markers, etc.

As is shown in the Examples below, the MRCs can form colonies in soft agar, which is indicative of clonogenicity. When placed in culture, the MRCs in some embodiments proliferate without feeder cells or conditioned medium, as is often required for cells that are not clonogenic or capable of repopulating cell populations.

MRCs can be isolated by a variety of methods known in the art. These methods include flow cytometry, such as fluorescence activated cell sorting. One preferred method is to contact a population of melanoma cells with an antibody that binds CD133 and isolate the contacted cells by flow cytometry. Typically the antibody used in such a method is detectably labeled, e.g., with a fluorescent molecule, to facilitate sorting of the cells bound by the antibody. Alternatively, the contacted cells are further contacted with a detectably labeled molecule that binds to the antibody.

Another method for separating cells to isolate MRCs or enrich MRCs in populations of cells is to use magnetic activated cell sorting. In one such method, MRCs are isolated by contacting a population of melanoma cells with immunomagnetic particles (e.g., beads) that include an antibody that binds to CD133, and then isolating the immunomagnetic particles. Still other methods for separating cells are known to the skilled person.

Thus the invention provides methods for isolating melanoma repopulating cells (MRCs) and enriching MRCs in cell populations. The methods generally include contacting a population of melanoma cells with one or more labeled agents, such as antibodies that bind to CD133 and/or a cancer-testis antigen (CTAg), and then isolating the MRCs based on the binding of the labeled antibodies to the MRCs. The population of melanoma cells can be any population of cells that is suspected of having MRCs. These may include populations of cells obtained from a tumor sample, such as a biopsy, or a cell line. The MRCs can be isolated for further study, for establishment of cultures, for preparing melanoma models of disease, and the like.

The isolated cell populations of the invention preferably are enriched for MRCs, i.e., have a higher proportion of MRCs than a native cell population (taken from a subject or tumor sample) or an unenriched population of cultured cells. In a preferred embodiment, the MRCs are enriched in the cell population by at least 2-fold relative to an unenriched cell population. More preferably, the MRCs are enriched in the cell population by at least 5-fold relative to an unenriched cell population. Still more preferably, the MRCs are enriched in the cell population by at least 10-fold relative to an unenriched cell population. Still more preferably, the MRCs are enriched in the cell population by at least 20-fold relative to an unenriched cell population. Yet more preferably, the MRCs are enriched in the cell population by at least 50-fold relative to an unenriched cell population.

The identification and characterization of MRCs (and optionally the isolation of MRCs and/or enrichment of MRCs in cell populations) also facilitates therapeutic and diagnostic methods based on the properties of the MRCs. These methods generally utilize the ability to selectively recognize MRCs and isolate the MRCs, kill the MRCs, or inhibit proliferation of the MRCs, thereby permitting treatment or diagnosis of cancer, particularly melanoma.

The invention provides for treatment of cancer in a subject and for killing or inhibiting proliferation of MRCs. The treatment of cancer in this manner can be a monotherapy, i.e., treating a patient only by using the methods and compositions described here to kill the MRCs or inhibit proliferation of the MRCs. However, the cancer treatment also can be an adjunct therapy to one or more other therapies, including immune therapies such as vaccination, surgery, radiation therapy, and chemotherapy. Aspects of these methods are described in greater detail elsewhere herein.

Particular methods for killing or inhibiting the proliferation of melanoma repopulating cells (MRCs), include contacting a population of melanoma cells (such as a melanoma tumor, cell line or cell sample such as a biopsy) with an agent or combination of agents selectively targeted to MRCs of the population of melanoma cells. By “selectively targeted” is meant that the agent or combination of agents selectively recognizes and binds to MRCs as compared to non-MRCs in a tissue or cell population. The agent or combination of agents can effectively kill the MRCs or inhibit the proliferation of MRCs by one of several mechanisms, such as by induction of apoptosis, bringing into close proximity a cytotoxic or cytostatic agent, or attracting other cells such as cytotoxic T lymphocytes or macrophages that can kill or inhibit proliferation of the MRCs. By “cytotoxic or cytostatic agent” is meant an agent (e.g., molecule) that kills or reduces proliferation of cells.

The agent in certain preferred embodiments is an antibody, or antigen-binding fragment thereof, which binds CD133 and/or a CTAg. The antibody or fragment can be a bispecific or multispecific antibody or fragment that binds CD133 and at least one CTAg. By “bispecific” is meant an agent (e.g., antibody or antigen-binding fragment thereof) that binds to two different antigens. By “multispecific” is meant an agent (e.g., antibody or antigen-binding fragment thereof) that binds to at least two different antigens, preferably at least three antigens.

In some cases, the labeled antibodies (or other agents known to those of skill in the art to selectively bind to proteins) are labeled with detectable molecules, preferably fluorescent molecules, or magnetic entities, such as magnetic particles. These molecules or entities can be used to facilitate detection and/or separation of the MRCs from other cells of a cell population. As such, MRCs can be isolated by a variety of methods known in the art, including preferred methods such as fluorescence-activated cell sorting and magnetic activated cell sorting.

Labeled agents (e.g., antibodies) can be used sequentially or simultaneously to isolate MRCs. In one exemplary method, melanoma cells are contacted first with labeled antibodies that bind to CD133, followed by contacting the cells with labeled antibodies that bind to a CTAg. In another exemplary method, melanoma cells are contacted first with labeled antibodies that bind to a CTAg, followed by contacting the cells with labeled antibodies that bind to CD133. In a third exemplary method, melanoma cells are contacted simultaneously with labeled antibodies that bind to CD133 and with labeled antibodies that bind to a CTAg. In each of these methods, the cells can be isolated after each labeling step, or can be isolated after labeling with both agents.

Diagnostic methods based on identification and characterization of MRCs include identifying the presence of melanoma repopulating cells (MRCs) in an animal or subject in vivo, ex vivo or in vitro. Such in vivo methods include administering to the animal a detectably labeled agent that binds to CD133 and/or a CTAg. For diagnostic procedures on internal cells, the detectably labeled agent circulates and binds to MRCs in the body of the animal or subject, thereby facilitating detection of MRCs. The diagnostic procedures also can be used for cells in skin, and this may include topical administration of detectably labeled agent that binds to CD133 and/or a CTAg. The agent also can be administered intratumorally, i.e., by direct injection into a tumor.

For ex vivo or in vitro methods, a cell sample, tissue sample or other population of cells suspected of having MRCs is contacted with a detectably labeled agent that binds to CD133 and/or a CTAg, and detection of binding of the detectably labeled agent indicates the presence of MRCs in the cell sample or other population of cells.

Identification of MRCs in the population can contribute to diagnosis or classification of a tumor as a melanoma. The methods therefore may be of use in identifying metastatic melanoma tumors.

The diagnostic agent in certain preferred embodiments is an antibody, or antigen-binding fragment thereof, which binds CD133 and/or a CTAg. The antibody or fragment can be a bispecific or multispecific antibody or fragment that binds CD133 and at least one CTAg.

The invention thus includes diagnosing or monitoring cancer in a subject by determining the presence or amount of MRCs, which express CD133 and preferably one or more cancer-testis antigens, more preferably NY-ESO-1. In preferred embodiments, this determination is performed by assaying a biological sample obtained from the subject, such as tumor biopsy (preferably a biopsy of a melanoma or cells suspected of being melanoma cells), cell scraping, serum, blood, or lymph node fluid, for the presence of MRCs as described herein. This determination may also be performed by assaying a tissue or cells from the subject for the presence of one or more cells expressing both CD133 and one or more cancer-testis antigens (or nucleic acid molecules that encode these antigens) described herein.

For example, the invention permits more accurate determinations of prognosis, based on the existence of MRCs in a tumor. A tumor with fewer or no MRCs would be expected to have less (or no) repopulation capacity, and therefore be less aggressive, metastatic, or able to withstand cancer therapy.

Measurement of the MRCs in a tumor over time by sequential determinations permits monitoring of the disease and/or the effects of a course of treatment. For example, a cell sample, such as a biopsy, may be obtained from a subject, tested for the existence or quantity of MRCs, and at a second, subsequent time, another sample, may be obtained from the subject and similarly tested. The results of the first and second (or subsequent) tests can be compared as a measure of the onset, regression or progression of cancer, or, if cancer treatment was undertaken during the interval between obtaining the samples, the effectiveness of the treatment may be evaluated by comparing the results of the two tests.

Diagnostic methods of the invention may involve determining the expression of one or more of markers of MRCs described herein, such as CD133 and/or cancer-testis antigens, or the nucleic acid molecules that encode them. Such determinations can be carried out via any standard nucleic acid assay, including the polymerase chain reaction or assaying with hybridization probes, which may be labeled, or by assaying biological samples with binding partners (e.g., antibodies) for cancer-testis antigens using standard methodologies.

The diagnostic methods of the invention can be used to detect the presence of a cancer associated with MRCs, such as melanoma, by determining the number of MRCs present in a tissue or cell sample, as well as to assess the progression and/or regression of the cancer such as in response to treatment (e.g., chemotherapy, radiation). It is expected that a typical non-cancer tissue or cell sample will have zero or a very low number of MRCs, whereas a cancer tissue or cell sample will have a significantly higher number of MRCs, which can be termed an “aberrant number” of MRCs. As used herein, aberrant numbers of MRCs is intended to include any number of MRCs that is different by a statistically significant amount from the expected amount of MRCs. For example, the presence of MRCs in a tissue that is not expected to have such cells would be an example of an “aberrant number” of MRCs. Likewise, a significantly higher number of MRCs than expected is another example of an “aberrant number” of MRCs. Therefore, a determination of the presence and/or amount amount of MRCs is diagnostic of cancer if the level of expression is above a baseline level determined for that tissue type. The baseline level of expression can be determined using standard methods known to those of skill in the art. Such methods include, for example, assaying a number of histologically normal tissue samples from subjects that are clinically normal (i.e., do not have clinical signs of cancer in that tissue type) and determining the mean level of MRCs for the samples.

The number of MRCs can indicate cancer in the tissue when the number of MRCs is significantly more in the tissue than in a negative control sample. In some embodiments, a number of MRCs in the tissue or cells being examined that is at least about 5%, 10%, 20%, 30%, 40%, 50%, 60%, 70%, 80%, 90%, 100%, 150%, 200%, 250%, 300%, 400%, or 500% more than the number of MRCs in negative control tissue or cells indicates cancer in the tissue or cells being examined.

As used herein the term “control” means samples of materials tested in parallel with the experimental materials. Examples include samples from control cell populations or control tissue samples generated through manufacture to be tested in parallel with the experimental samples.

Typically the negative control will be based on apparently healthy individuals in an appropriate age bracket. Positive control cell populations or tissue samples, i.e., that contain MRCs, can be used to verify experimental procedures.

Determining the presence of MRCs can, in some aspects, be performed by determining the level (i.e., presence or amount) of expression of one or more MRC marker nucleic acid molecules and/or polypeptides (such as CD133 and/or one of more CTAgs). The expression of one or more MRC markers may be determined using routine methods known to those of ordinary skill in the art. These methods include, but are not limited to: direct RNA amplification, reverse transcription of RNA to cDNA, real-time RT-PCR, amplification of cDNA, hybridization, and immunologically based assay methods, which include, but are not limited to immunohistochemistry, antibody sandwich capture assay, ELISA, and enzyme-linked immunospot assay (EliSpot assay). For example, the determination of the presence or level of one or more MRC marker nucleic acid molecules in a subject or tissue can be carried out via any standard nucleic acid determination assay, including the polymerase chain reaction, or assaying with labeled hybridization probes. Such hybridization methods include, but are not limited to, microarray techniques.

These methods of determining the presence and/or level of the one or more MRC markers in cells and tissues may include use of labels to monitor the presence of the MRCs. Such labels may include, but are not limited to radiolabels or chemiluminescent labels, which may be utilized to determine whether one or more MRC markers is expressed in a cell or tissue, and to determine the level of expression in the cell or tissue. For example, as described elsewhere herein, a fluorescently labeled or radiolabeled antibody that selectively binds to one or more MRC markers may be contacted with a tissue or cell to visualize the polypeptide in vitro or in vivo. These and other in vitro and in vivo imaging methods for determining the presence of the one or more MRC markers are well known to those of ordinary skill in the art.

The invention includes kits for assaying the presence of one or more MRC markers, preferably antibodies that specifically bind to one or more MRC markers. An example of such a kit may include an antibody, or antigen-binding fragment thereof, that binds specifically to one or more MRC markers, such as CD133 and/or one or more CTAgs. The antibody or antigen-binding fragment thereof, may be applied to a tissue or cell sample from a patient with cancer and the sample then processed to assess whether specific binding occurs between the antibody and one or more MRC markers. In addition, the antibody or antigen-binding fragment thereof may be applied to a body fluid sample, such as serum, from a subject, either suspected of having cancer, diagnosed with cancer, or believed to be free of cancer. As will be understood by one of skill in the art, such binding assays may also be performed with a sample or object contacted with an antibody and/or cancer-testis antigen that is in solution, for example in a 96-well plate or applied directly to an object surface.

Another example of a kit of the invention is a kit that provides components necessary to determine the level of expression of one or more one or more MRC markers. Such components may include primers useful for amplification of one or more MRC markers and/or other chemicals for PCR amplification. Another example of a kit of the invention is a kit that provides components necessary to determine the level of expression of one or more MRC markers using a method of hybridization.

The foregoing kits can include instructions or other printed material on how to use the various components of the kits for diagnostic purposes.

As used herein, a “subject” is preferably a human, non-human primate, cow, horse, pig, sheep, goat, dog, cat or rodent. In all embodiments, human subjects are preferred. In some embodiments, the subject is suspected of having cancer or has been diagnosed with cancer.

As used herein, a “biological sample” includes, but is not limited to: tissue, cells and/or body fluid (e.g. serum, blood, lymph node fluid, etc.). The fluid sample may include cells and/or fluid. The tissue and cells may be obtained from a subject or may be grown in culture (e.g. from a cell line). As used herein, a biological sample is body fluid, tissue or cells obtained from a subject using methods well-known to those of ordinary skill in the related medical arts.

As used herein, the “nucleic acid molecules that encode” means the nucleic acid molecules that code for the one or more MRC markers. These nucleic acid molecules may be DNA or may be RNA (e.g. mRNA). The one or more MRC markers also encompass variants of the molecules described herein. These variants may be splice variants or allelic variant, as are known in the art. Variants of the nucleic acid molecules for use as described herein are intended to include homologs and alleles. In all embodiments, human MRC markers (including CD133 and cancer-testis antigens) and the encoding nucleic acid molecules thereof, are preferred.

As used herein the term “isolated nucleic acid molecule” means: (i) amplified in vitro by, for example, polymerase chain reaction (PCR); (ii) recombinantly produced by cloning; (iii) purified, as by cleavage and gel separation; or (iv) synthesized by, for example, chemical synthesis. An isolated nucleic acid is one which is readily manipulable by recombinant DNA techniques well known in the art. Thus, a nucleotide sequence contained in a vector in which 5′ and 3′ restriction sites are known or for which polymerase chain reaction (PCR) primer sequences have been disclosed is considered isolated but a nucleic acid sequence existing in its native state in its natural host is not. An isolated nucleic acid may be substantially purified, but need not be. For example, a nucleic acid that is isolated within a cloning or expression vector is not pure in that it may comprise only a tiny percentage of the material in the cell in which it resides. Such a nucleic acid is isolated, however, as the term is used herein because it is readily manipulable by standard techniques known to those of ordinary skill in the art.

Optimal alignment of sequences for comparison may be conducted using programs such as BLAST, publicly available on the National Library of Medicine website. Other programs such as UniGene (The National Library of Medicine website), SAGE Anatomic Reviewer and its Virtual Northern tool, (The Cancer Genome Anatomy Project CGAP website) are also publicly available. Preferably, the “percentage of sequence identity” is determined by comparing two optimally aligned sequences over a window of comparison of at least 20 positions, wherein the portion of the polynucleotide or polypeptide sequence in the comparison window may comprise additions or deletions (i.e., gaps) of 20 percent or less, usually 5 to 15 percent, or 10 to 12 percent, as compared to the reference sequences (which to does not comprise additions or deletions) for optimal alignment of the two sequences. The percentage is calculated by determining the number of positions at which the identical nucleic acid bases or amino acid residue occurs in both sequences to yield the number of matched positions, dividing the number of matched positions by the total number of positions in the reference sequence (i.e., the window size) and multiplying the results by 100 to yield the percentage of sequence identity.

In general, homologs, alleles and preferred variants of the one or more MRC markers typically will share at least 90% nucleotide identity and/or at least 95% amino acid identity to the sequences of cancer-testis nucleic acids and polypeptides, respectively, in some instances will share at least 95% nucleotide identity and/or at least 97% amino acid identity, in other instances will share at least 97% nucleotide identity and/or at least 98% amino acid identity, in other instances will share at least 99% nucleotide identity and/or at least 99% amino acid identity, and in other instances will share at least 99.5% nucleotide identity and/or at least 99.5% amino acid identity.

According to yet another aspect of the invention, an expression vector comprising any of the MRC markers, preferably operably linked to a promoter, is provided. In a related aspect, host cells transformed or transfected with such expression vectors also are provided. As used herein, a “vector” may be any of a number of nucleic acid molecules into which a desired sequence may be inserted by restriction and ligation for transport between different genetic environments or for expression in a host cell. Vectors are typically composed of DNA although RNA vectors are also available. Vectors include, but are not limited to, plasmids, phagemids, and virus genomes. A cloning vector is one which is able to replicate in a host cell, and which is further characterized by one or more endonuclease restriction sites at which the vector may be cut in a determinable fashion and into which a desired DNA sequence may be ligated such that the new recombinant vector retains its ability to replicate in the host cell. In the case of plasmids, replication of the desired sequence may occur many times as the plasmid increases in copy number within the host bacterium or just a single time per host before the host reproduces by mitosis. In the case of phage, replication may occur actively during a lytic phase or passively during a lysogenic phase. An expression vector is one into which a desired DNA sequence may be inserted by restriction and ligation such that it is operably joined to regulatory sequences and may be expressed as an RNA transcript. Vectors may further contain one or more marker sequences suitable for use in the identification of cells which have or have not been transformed or transfected with the vector. Markers include, for example, genes encoding proteins which increase or decrease either resistance or sensitivity to antibiotics or other compounds, genes which encode enzymes whose activities are detectable by standard assays known in the art, e.g., beta-galactosidase or alkaline phosphatase, and genes which visibly affect the phenotype of transformed or transfected cells, hosts, colonies or plaques, e.g., green fluorescent protein. Preferred vectors are those capable of autonomous replication and expression of the structural gene products present in the DNA segments to which they are operably joined.

As used herein, a coding sequence and regulatory sequences are said to be “operably joined” when they are covalently linked in such a way as to place the expression or transcription of the coding sequence under the influence or control of the regulatory sequences. As used herein, “operably joined” and “operably linked” are used interchangeably and should be construed to have the same meaning. If it is desired that the coding sequences be translated into a functional protein, two DNA sequences are said to be operably joined if induction of a promoter in the 5′ regulatory sequences results in the transcription of the coding sequence and if the nature of the linkage between the two DNA sequences does not (1) result in the introduction of a frame-shift mutation, (2) interfere with the ability of the promoter region to direct the transcription of the coding sequences, or (3) interfere with the ability of the corresponding RNA transcript to be translated into a protein. Thus, a promoter region is operably joined to a coding sequence if the promoter region is capable of effecting transcription of that DNA sequence such that the resulting transcript can be translated into the desired protein or polypeptide.

The precise nature of the regulatory sequences needed for gene expression may vary between species or cell types, but shall in general include, as necessary, 5′ non-transcribed and 5′ non-translated sequences involved with the initiation of transcription and translation respectively, such as a TATA box, capping sequence, CAAT sequence, and the like. Often, such 5′ non-transcribed regulatory sequences will include a promoter region which includes a promoter sequence for transcriptional control of the operably joined gene. Regulatory sequences may also include enhancer sequences or upstream activator sequences as desired. The vectors of the invention may optionally include 5′ leader or signal sequences. The choice and design of an appropriate vector is within the ability and discretion of one of ordinary skill in the art.

It will also be recognized that the invention embraces the use of the one or more MRC markers and genomic sequences in expression vectors, as well as to transfect host cells and cell lines, be these prokaryotic, e.g., E. coli, or eukaryotic, e.g., CHO cells, COS cells, yeast expression systems, and recombinant baculovirus expression in insect cells. Especially useful are mammalian cells such as human, mouse, hamster, pig, goat, primate, etc. They may be of a wide variety of tissue types, including mast cells, fibroblasts, oocytes, and lymphocytes, and may be primary cells and cell lines. Specific examples include dendritic cells, peripheral blood leukocytes, bone marrow stem cells and embryonic stem cells. The expression vectors require that the pertinent sequence, i.e., those nucleic acids described supra, be operably linked to a promoter.

The invention, in one aspect, also permits the construction of “knock-outs” and “knock-ins” in cells and in animals of one or more of the MRC marker genes, providing materials for studying certain aspects of cancer and immune system responses to cancer.

Expression vectors containing all the necessary elements for expression are commercially available and known to those skilled in the art. Cells are genetically engineered by the introduction into the cells of heterologous DNA or RNA encoding one or more MRC markers, fragments, or variants thereof. The heterologous DNA or RNA is placed under operable control of transcriptional elements to permit the expression of the heterologous DNA in the host cell.

Preferred systems for mRNA expression in mammalian cells are those such as pcDNA/V5-GW/D-TOPO® and pcDNA3.1 (Invitrogen) that contain a selectable marker (which facilitates the selection of stably transfected cell lines) and contain the human cytomegalovirus (CMV) enhancer-promoter sequences. Additionally, suitable for expression in primate or canine cell lines is the pCEP4 vector (Invitrogen), which contains an Epstein Barr virus (EBV) origin of replication, facilitating the maintenance of plasmid as a multicopy extrachromosomal element.

The invention also involves the use of agents such as polypeptides that bind to one or more MRC markers, such as CD133 and CTAgs. Such agents can be used in methods of the invention including the diagnosis and/or treatment of cancer. Such binding agents can be used, for example, in screening assays to detect the presence or absence of MRCs and can be used in quantitative assays to determine numbers of MRCs in biological samples and cells. Such agents also may be used to inhibit the native activity of the one or more MRC markers, for example, by binding to such polypeptides.

In preferred embodiments, the binding polypeptide is an antibody or antibody fragment, more preferably, an Fab or F(ab)₂ fragment of an antibody. Typically, the fragment includes a CDR3 region that is selective for the one or more MRC markers. Any of the various types of antibodies can be used for this purpose, including polyclonal antibodies, monoclonal antibodies, humanized antibodies, and chimeric antibodies.

Thus, the invention provides agents which bind to one or more MRC markers. Such binding partners can be used in screening assays to detect the presence or absence of MRCs and in purification protocols to isolate such MRCs. Likewise, such binding partners can be used to selectively target drugs, toxins or other molecules (including detectable diagnostic molecules) to cells which express one or more MRC markers. In this manner, for example, cells present in solid or non-solid tumors that express one or more MRC markers can be treated with cytotoxic compounds that are selective for the one or more MRC markers (nucleic acids and/or antigens). Such binding agents also can be used to inhibit the native activity of the one or more MRC markers, for example, to further characterize the functions of these molecules.

The antibodies of the present invention are prepared by any of a variety of methods, including administering a protein, fragments of a protein, cells expressing the protein or fragments thereof and the like to an animal to induce polyclonal antibodies. The production of monoclonal antibodies is well known in the art. As detailed herein, such antibodies may be used, for example, to identify MRCs in tissues or to purify MRCs. Antibodies also may be coupled to specific labeling agents or imaging agents, including, but not limited to a molecule preferably selected from the group consisting of fluorescent, enzyme, radioactive, metallic, biotin, chemiluminescent, bioluminescent, chromophore, or colored, etc. In some aspects of the invention, a label may be a combination of the foregoing molecule types.

Significantly, as is well-known in the art, only a small portion of an antibody molecule, the paratope, is involved in the binding of the antibody to its epitope (see, in general, Clark, W. R. (1986) The Experimental Foundations of Modern Immunology Wiley & Sons, Inc., New York; Roitt, (1991) Essential Immunology, 7th Ed., Blackwell Scientific Publications, Oxford). The pFc′ and Fc regions, for example, are effectors of the complement cascade but are not involved in antigen binding. An antibody from which the pFc′ region has been enzymatically cleaved, or which has been produced without the pFc′ region, designated an F(ab′)2 fragment, retains both of the antigen binding sites of an intact antibody. Similarly, an antibody from which the Fc region has been enzymatically cleaved, or which has been produced without the Fc region, designated an Fab fragment, retains one of the antigen binding sites of an intact antibody molecule. Fab fragments consist of a covalently bound antibody light chain and a portion of the antibody heavy chain denoted Fd. The Fd fragments are the major determinant of antibody specificity (a single Fd fragment may be associated with up to ten different light chains without altering antibody specificity) and Fd fragments retain epitope-binding ability in isolation.

Within the antigen-binding portion of an antibody, as is well-known in the art, there are complementarity determining regions (CDRs), which directly interact with the epitope of the antigen, and framework regions (FRs), which maintain the tertiary structure of the paratope (see, in general, Clark, 1986; Roitt, 1991). In both the heavy chain Fd fragment and the light chain of IgG immunoglobulins, there are four framework regions (FR1 through FR4) separated respectively by three complementarity determining regions (CDR1 through CDR3). The CDRs, and in particular the CDR3 regions, and more particularly the heavy chain CDR3, are largely responsible for antibody specificity.

It is now well-established in the art that the non-CDR regions of a mammalian antibody may be replaced with similar regions of nonspecific or heterospecific antibodies while retaining the epitopic specificity of the original antibody. This is most clearly manifested in the development and use of “humanized” antibodies in which non-human CDRs are covalently joined to human FR and/or Fc/pFc′ regions to produce a functional antibody. See, e.g., U.S. Pat. Nos. 4,816,567, 5,225,539, 5,585,089, 5,693,762, and 5,859,205.

Fully human monoclonal antibodies also can be prepared by immunizing mice transgenic for large portions of human immunoglobulin heavy and light chain loci. Following immunization of these mice (e.g., XenoMouse (Abgenix), HuMAb mice (Medarex/GenPharm)), monoclonal antibodies can be prepared according to standard hybridoma technology. These monoclonal antibodies will have human immunoglobulin amino acid sequences and therefore will not provoke human anti-mouse antibody (HAMA) responses when administered to humans.

Thus, as will be apparent to one of ordinary skill in the art, the present invention also provides for F(ab′)₂, Fab, Fv, and Fd fragments; chimeric antibodies in which the Fc and/or FR and/or CDR1 and/or CDR2 and/or light chain CDR3 regions have been replaced by homologous human or non-human sequences; chimeric F(ab′)2 fragment antibodies in which the FR and/or CDR1 and/or CDR2 and/or light chain CDR3 regions have been replaced by homologous human or non-human sequences; chimeric Fab fragment antibodies in which the FR and/or CDR1 and/or CDR2 and/or light chain CDR3 regions have been replaced by homologous human or non-human sequences; and chimeric Fd fragment antibodies in which the FR and/or CDR1 and/or CDR2 regions have been replaced by homologous human or non-human sequences. The present invention also includes so-called single chain antibodies (e.g., ScFv), (single) domain antibodies, and other intracellular antibodies. Domain antibodies, camelid and camelized antibodies and fragments thereof, such as those described in patents and published patent applications of Ablynx N V and Domantis also can be used as described herein.

Thus, the invention involves polypeptides of numerous size and type that bind specifically to one or more MRC markers. These polypeptides may be derived also from sources other than antibody technology. For example, such polypeptide binding agents can be provided by degenerate peptide libraries which can be readily prepared in solution, in immobilized form or as phage display libraries. Combinatorial libraries also can be synthesized of peptides containing one or more amino acids. Libraries further can be synthesized of peptides and non-peptide synthetic moieties.

The one or more MRC markers can be used to screen peptide libraries, including phage display libraries, to identify and select peptide binding partners of the one or more MRC markers. Such molecules can be used, as described herein, for screening assays, for diagnostic assays, for purification protocols or for targeting drugs, toxins and/or labeling agents (e.g., radioisotopes, fluorescent molecules, etc.) to MRCs.

Phage display can be particularly effective in identifying binding agents useful according to the invention. Briefly, one prepares a phage library (using e.g. m13, fd, or lambda phage), displaying inserts from 4 to about 80 amino acid residues using conventional procedures. The inserts may represent, for example, a completely degenerate or biased array. One then can select phage-bearing inserts which bind to one or more MRC markers. This process can be repeated through several cycles of reselection of phage that bind to the one or more MRC markers. Repeated rounds lead to enrichment of phage bearing particular sequences. DNA sequence analysis can be conducted to identify the sequences of the expressed polypeptides. The minimal linear portion of the sequence that binds to the one or more MRC markers can be determined. One can repeat the procedure using a biased library containing inserts containing part or all of the minimal linear portion plus one or more additional degenerate residues upstream or downstream thereof. Yeast two-hybrid screening methods also may be used to identify polypeptides that bind to the one or more MRC markers, and thereby to MRCs.

As detailed herein, the foregoing antibodies, fragments and other binding agents may be used to identify tissues with normal or aberrant expression of a cancer-testis antigen. Antibodies also may be coupled to specific diagnostic labeling agents for imaging of MRCs in cells and tissues or to therapeutically useful agents according to standard coupling procedures. As used herein, “therapeutically useful agents” include any therapeutic molecule which desirably is targeted selectively to MRCs.

Diagnostic agents for in vivo use include various contrast agents including, but are not limited to, barium sulfate, iocetamic acid, iopanoic acid, ipodate calcium, diatrizoate sodium, diatrizoate meglumine, metrizamide, tyropanoate sodium and radiodiagnostics including positron emitters such as fluorine-18 and carbon-11, gamma emitters such as iodine-123, technitium-99, iodine-131 and indium-111, and nuclides for nuclear magnetic resonance such as fluorine and gadolinium. Other diagnostic agents useful in the invention will be apparent to one of ordinary skill in the art.

The antibodies of the present invention can also be used to therapeutically target MRCs. In a preferred embodiment, antibodies can be used to target one or more MRC markers expressed on the cell surface. These antibodies can be linked not only to a detectable marker but also an antitumor agent or an immunomodulator. Antitumor agents can include cytotoxic agents and agents that act on tumor neovasculature. Detectable markers include, for example, radioactive or fluorescent markers. Cytotoxic agents include cytotoxic radionuclides, chemical toxins and protein toxins.

The cytotoxic radionuclide or radiotherapeutic isotope preferably is an alpha-emitting isotope such as ²²⁵ Ac, ²¹¹At, ²¹²Bi, ²¹³Bi, ²¹²Pb, ²²⁴Ra or ²²³Ra. Alternatively, the cytotoxic radionuclide may a beta-emitting isotope such as ¹⁸⁶Rh, ¹⁸⁸Rh, ¹⁷⁷Lu, ⁹⁰Y, ¹³¹I, ⁶⁷Cu, ⁶⁴Cu, ¹⁵³Sm or ¹⁶⁶Ho. Further, the cytotoxic radionuclide may emit Auger and low energy electrons and include the isotopes ¹²⁵I, ¹²³I or ⁷⁷Br.

Suitable chemical toxins or chemotherapeutic agents include members of the enediyne family of molecules, such as calicheamicin and esperamicin. Chemical toxins can also be taken from the group consisting of methotrexate, doxorubicin, melphalan, chlorambucil, ARA-C, vindesine, mitomycin C, cis-platinum, etoposide, bleomycin and 5-fluorouracil. Other antineoplastic agents that may be conjugated to the antibodies of the present invention include dolastatins (U.S. Pat. Nos. 6,034,065 and 6,239,104) and derivatives thereof. Of particular interest is dolastatin 10 (dolavaline-valine-dolaisoleuine-dolaproine-dolaphenine) and the derivatives auristatin PHE (dolavaline-valine-dolaisoleuine-dolaproine-phenylalanine-methyl ester) (Pettit, G. R. et al., Anticancer Drug Des. 13(4):243-277, 1998; Woyke, T. et al., Antimicrob. Agents Chemother. 45(12):3580-3584, 2001), and aurastatin E and the like. Toxins that are less preferred in the compositions and methods of the invention include poisonous lectins, plant toxins such as ricin, abrin, modeccin, botulina and diphtheria toxins. Of course, combinations of the various toxins could also be coupled to one antibody molecule thereby accommodating variable cytotoxicity. Other chemotherapeutic agents are known to those skilled in the art.

Agents that act on the tumor vasculature can include tubulin-binding agents such as combrestatin A4 (Griggs et al., Lancet Oncol. 2:82, 2001), angiostatin and endostatin (reviewed in Rosen, Oncologist 5:20, 2000, incorporated by reference herein) and interferon inducible protein 10 (U.S. Pat. No. 5,994,292). A number of antiangiogenic agents currently in clinical trials are also contemplated. Agents currently in clinical trials include: 2ME2, Angiostatin, Angiozyme, Anti-VEGF RhuMAb, Apra (CT-2584), Avicine, Benefin, BMS275291, Carboxyamidotriazole, CC4047, CC5013, CC7085, CDC801, CGP-41251 (PKC 412), CM101, Combretastatin A-4 Prodrug, EMD 121974, Endostatin, Flavopiridol, Genistein (GCP), Green Tea Extract, IM-862, ImmTher, Interferon alpha, Interleukin-12, Iressa (ZD1839), Marimastat, Metastat (Col-3), Neovastat, Octreotide, Paclitaxel, Penicillamine, Photofrin, Photopoint, PI-88, Prinomastat (AG-3340), PTK787 (ZK22584), RO317453, Solimastat, Squalamine, SU 101, SU 5416, SU-6668, Suradista (FCE 26644), Suramin (Metaret), Tetrathiomolybdate, Thalidomide, TNP-470 and Vitaxin. Additional antiangiogenic agents are described by Kerbel, J. Clin. Oncol. 19(18s):45s-51s, 2001, which is incorporated by reference herein. Immunomodulators suitable for conjugation to the antibodies include α-interferon, γ-interferon, and tumor necrosis factor alpha (TNFα).

The coupling of one or more toxin molecules to the antibody is envisioned to include many chemical mechanisms, for instance covalent binding, affinity binding, intercalation, coordinate binding, and complexation. The toxic compounds used to prepare the immunotoxins are attached to the antibodies or antigen-binding fragments thereof by standard protocols known in the art.

As described herein, the one or more MRC markers and the antibodies and other binding molecules, as described herein, can be used for the diagnosis, determination of prognosis and treatment of disorders. When “disorder” is used herein, it refers to any pathological condition where there are MRCs. An example of such a disorder is cancer. For human cancers, additional particular examples include, biliary tract cancer; bladder cancer; breast cancer; brain cancer including glioblastomas and medulloblastomas; cervical cancer; choriocarcinoma; colon cancer including colorectal carcinomas; endometrial cancer; esophageal cancer; gastric cancer; head and neck cancer; hematological neoplasms including acute lymphocytic and myelogenous leukemia, multiple myeloma, AIDS-associated leukemias and adult T-cell leukemia lymphoma; intraepithelial neoplasms including Bowen's disease and Paget's disease; liver cancer; lung cancer including small cell lung cancer and non-small cell lung cancer; lymphomas including Hodgkin's disease and lymphocytic lymphomas; neuroblastomas; oral cancer including squamous cell carcinoma; osteosarcomas; ovarian cancer including those arising from epithelial cells, stromal cells, germ cells and mesenchymal cells; pancreatic cancer; prostate cancer; rectal cancer; sarcomas including leiomyosarcoma, rhabdomyosarcoma, liposarcoma, fibrosarcoma, synovial sarcoma, neurosarcoma, chondrosarcoma, Ewing sarcoma, malignant fibrous histocytoma, glioma, esophageal cancer, hepatoma and osteosarcoma; skin cancer including melanomas, Kaposi's sarcoma, basocellular cancer, and squamous cell cancer; testicular cancer including germinal tumors such as seminoma, non-seminoma (teratomas, choriocarcinomas), stromal tumors, and germ cell tumors; testicular cancer; thyroid cancer including thyroid adenocarcinoma and medullar carcinoma; transitional cancer and renal cancer including adenocarcinoma and Wilms tumor.

Conventional treatment for cancer may include, but is not limited to: surgical intervention, chemotherapy, radiotherapy, and adjuvant systemic therapies. In one aspect of the invention, treatment may include administering binding polypeptides such as antibodies that specifically bind to the one or more MRC markers. These binding polypeptides can be optionally linked to one or more detectable markers, antitumor agents or immunomodulators as described above.

Cancer treatment, in another aspect of the invention, includes administering an antisense molecules or RNAi molecules to reduce expression level and/or function level of one or more MRC markers in the subject in cancers where MRCs have been identified. The use of RNA interference or “RNAi” involves the use of double-stranded RNA (dsRNA) to block gene expression. (see: Sui, G, et al, Proc Natl. Acad. Sci. U.S.A. 99:5515-5520, 2002). Methods of applying RNAi strategies in embodiments of the invention would be understood by one of ordinary skill in the art.

Methods in which small interfering RNA (siRNA) molecules are used to reduce the expression of one or more MRC markers may be used. In one aspect, a cell is contacted with a siRNA molecule to produce RNA interference (RNAi) that reduces expression of one or more MRC markers. The siRNA molecule is directed against nucleic acids coding for the one or more MRC markers (e.g. RNA transcripts including untranslated and translated regions). In a preferred aspect of the invention the MRC markers are CD133 and/or one or more CTAgs, preferably NY-ESO-1. The expression level of the targeted MRC markers can be determined using well known methods such as FACS or Western blotting for determining the level of protein expression and Northern blotting or RT-PCR. for determining the level of mRNA transcript of the target gene.

As used herein, a “siRNA molecule” is a double stranded RNA molecule (dsRNA) consisting of a sense and an antisense strand or a single stranded molecule that has a dsRNA component, for example a section of the molecule that hybridizes to itself (e.g., a “hairpin” structure). The antisense strand of the siRNA molecule is a complement of the sense strand (Tuschl, T. et al., 1999, Genes & Dev., 13:3191-3197; Elbashir, S. M. et al., 2001, EMBO J., 20:6877-6888; incorporated herein by reference). In one embodiment the last nucleotide at the 3′ end of the antisense strand may be any nucleotide and is not required to be complementary to the region of the target gene. The siRNA molecule may be 19-23 nucleotides in length and form a hairpin structure. In one preferred embodiment the siRNA molecule includes a two nucleotide 3′ overhang on the sense strand. In a second preferred embodiment the two nucleotide overhang is thymidine-thymidine (TT). The siRNA molecule corresponds to at least a portion of a target gene. In one embodiment the siRNA molecule corresponds to a region selected from a cDNA target gene beginning between 50 to 100 nucleotides downstream of the start codon. In a preferred embodiment the first-nucleotide of the siRNA molecule is a purine.

The siRNA molecules can be plasmid-based. In a preferred method, a nucleic acid sequence that encodes a MRC marker is amplified using the well known technique of polymerase chain reaction (PCR). The use of the entire polypeptide encoding sequence is not necessary; as is well known in the art, a portion of the polypeptide encoding sequence is sufficient for RNA interference. The PCR fragment is inserted into a vector using routine techniques well known to those of skill in the art. In one aspect the nucleotide encoding sequence is the coding sequence of NY-ESO-1. In another preferred aspect the nucleotide encoding sequence is the coding sequence of CD133. Combinations of the foregoing can be expressed from a single vector or from multiple vectors introduced into cells.

In one aspect of the invention a mammalian vector comprising any of the MRC markers coding sequences is provided. The mammalian vectors include but are not limited to the pSUPER RNAi vectors (Brummelkamp, T. R. et al., 2002, Science, 296:550-553, incorporated herein by reference). In one embodiment a nucleotide coding sequence can be inserted into the mammalian vector using restriction sites, creating a stem-loop structure. In a second embodiment, the mammalian vector may comprise the polymerase-III H1-RNA gene promoter. The polymerase-III H1-RNA promoter produces a RNA transcript lacking a polyadenosine tail and has a well-defined start of transcription and a termination signal consisting of five thymidines (T5) in a row. The cleavage of the transcript at the termination site occurs after the second uridine and yields a transcript resembling the ends of synthetic siRNAs containing two 3′ overhanging T or U nucleotides. The antisense strand of the siRNA molecule hybridizes to the corresponding region of the mRNA of the target gene.

Preferred systems for mRNA expression in mammalian cells are those such as pSUPER RNAi system as described in Brummelkamp et al. (2002, Science, 296:550-553). Other examples include but are not limited to pSUPER.neo, pSUPER.neo+gfp, pSUPER.puro, BLOCK-iT T7-TOPO linker, pcDNA1.2/V5-GW/laCZ, pENTR/U6, pLenti6-GW/U6-laminshma, and pLenti6/BLOCK-iT-DEST. These vectors are available from suppliers such as Invitrogen, and one of skill in the art would be able to obtain and use them.

MRC markers can also be used in one aspect of the invention to induce or enhance an immune response. Some therapeutic approaches based upon the disclosure are premised on a response by a subject's immune system, leading to lysis of antigen presenting cells, such as MRCs which present one or more MRC markers. One such approach is the administration of autologous CTLs specific to a MRC marker/MHC complex to a subject with MRCs. It is within the ability of one of ordinary skill in the art to develop such CTLs in vitro. An example of a method for T cell differentiation is presented in International Application number PCT/US96/05607. Generally, a sample of cells taken from a subject, such as blood cells, are contacted with a cell presenting the complex and capable of provoking CTLs to proliferate. The target cell can be a transfectant, such as a COS cell. Alternatively, instead of transfecting COS cells, one might use autologous APCs such as dendritic cells (DCs) purified from peripheral blood mononuclear cells (PBMC). DCs could be transfected or pulsed with antigen, either full length protein or peptide antigens. (Ayyoub, M et al J. Immunol. 2004 172:7206-7211, Ayyoub M. et al. J Clin Invest 2004 113:1225-33.) These transfectants present the desired complex of their surface and, when combined with a CTL of interest, stimulate its proliferation. COS cells are widely available, as are other suitable host cells. Specific production of CTL clones is well known in the art. The clonally expanded autologous CTLs then are administered to the subject.

Another method for selecting antigen-specific CTL clones has been described (Altman et al., Science 274:94-96, 1996; Dunbar et al., Curr. Biol. 8:413-416, 1998), in which fluorogenic tetramers or multimers of MHC class I molecule/peptide complexes are used to detect specific CTL clones. Briefly, soluble MHC class I molecules are folded in vitro in the presence of β₂-microglobulin and a peptide antigen which binds the class I molecule. After purification, the MHC/peptide complex is purified and labeled with biotin. Tetramers are formed by mixing the biotinylated peptide-MHC complex with labeled avidin (e.g., phycoerythrin) at a molar ratio or 4:1. Tetramers are then contacted with a source of CTLs such as peripheral blood or lymph node. The tetramers bind CTLs which recognize the peptide antigen/MHC class I complex. Cells bound by the tetramers can be sorted by fluorescence activated cell sorting to isolate the reactive CTLs. The isolated CTLs then can be expanded in vitro for use as described herein.

The use of MHC class II molecules as tetramers was demonstrated by Crawford et al. (Immunity 8:675-682, 1998; see also Dunbar and Ogg, J. Immunol. Methods 268(1):3-7, 2002; Arnold et al., J. Immunol. Methods 271(1-2):137-151, 2002). Multimeric soluble MHC class II molecules were complexed with a covalently attached peptide (which can be attached with or without a linker molecule), but peptides also can be loaded onto class II molecules. The class II tetramers were shown to bind with appropriate specificity and affinity to specific T cells. Thus tetramers can be used to monitor both CD4⁺ and CD8⁺ cell responses to vaccination protocols. Methods for preparation of multimeric complexes of MHC class II molecules are described in Hugues et al., J. Immunological Meth. 268: 83-92 (2002) and references cited therein, each of which is incorporated by reference for such teachings.

In one therapeutic methodology, referred to as adoptive transfer (Greenberg, J. Immunol. 136(5): 1917, 1986; Riddel et al, Science 257: 238, 1992; Lynch et al, Eur. J. Immunol. 21: 1403-1410, 1991; Kast et al., Cell 59: 603-614, 1989), cells presenting the desired complex (e.g., dendritic cells) are combined with CTLs leading to proliferation of the CTLs specific thereto. The proliferated CTLs are then administered to a subject with a cellular abnormality which is characterized by certain of the abnormal cells presenting the to particular complex, such as MRCs presenting CTAg peptides, particularly NY-ESO-1 peptides. The CTLs then lyse the abnormal cells, thereby achieving the desired therapeutic goal.

The foregoing therapy assumes that at least some of the subject's abnormal cells present the relevant HLA/cancer associated antigen complex. This can be determined very easily, as the art is very familiar with methods for identifying cells which present a particular HLA molecule, as well as how to identify cells expressing DNA of the pertinent sequences, in this case a MRC marker sequence. Once cells presenting the relevant complex are identified via the foregoing screening methodology, they can be combined with a sample from a patient, where the sample contains CTLs. If the complex presenting cells are lysed by the mixed CTL sample, then it can be assumed that a MRC marker is being presented, and the subject is an appropriate candidate for the therapeutic approaches set forth supra.

Adoptive transfer is not the only form of therapy that is available in accordance with the invention. CTLs can also be provoked in vivo, using a number of approaches. One approach is the use of non-proliferative cells expressing the complex. The cells used in this approach may be those that normally express the complex, such as irradiated MRCs isolated as described herein or cells transfected with one or both of the genes necessary for presentation of the complex (i.e. the antigenic peptide and the presenting MHC molecule). Chen et al. (Proc. Natl. Acad. Sci. USA 88: 110-114, 1991) exemplifies this approach, showing the use of transfected cells expressing HPV E7 peptides in a therapeutic regime. Various cell types may be used. Similarly, vectors carrying one or both of the genes of interest may be used. Viral or bacterial vectors are especially preferred. For example, nucleic acids which encode a cancer-testis polypeptide may be operably linked to promoter and enhancer sequences which direct expression of the MRC marker polypeptide in certain tissues or cell types. The nucleic acid may be incorporated into an expression vector.

Expression vectors may be unmodified extrachromosomal nucleic acids, plasmids or viral genomes constructed or modified to enable insertion of exogenous nucleic acids, such as those encoding one or more MRC markers, as described elsewhere herein. Nucleic acids encoding one or more MRC markers also may be inserted into a retroviral genome, thereby facilitating integration of the nucleic acid into the genome of the target tissue or cell type. In these systems, the gene of interest is carried by a microorganism, e.g., a Vaccinia virus, pox virus, herpes simplex virus, retrovirus or adenovirus, and the materials de facto “infect” host cells. The cells which result present the complex of interest, and are recognized by autologous CTLs, which then proliferate.

A similar effect can be achieved by combining the MRC marker polypeptide or a stimulatory fragment thereof with an adjuvant to facilitate incorporation into antigen presenting cells in vivo. The MRC marker polypeptide is processed to yield the peptide partner of the MHC molecule while a MRC marker fragment may be presented without the need for further processing. Generally, subjects can receive an intradermal, intravenous, subcutaneous or intramuscular injection of an effective amount of the one or more MRC markers, e.g., CD133 or CTAgs, preferably NY-ESO-1. Initial doses can be followed by bi- or tri-weekly, weekly or monthly booster doses, following immunization protocols standard in the art. Preferred MRC markers include those where evidence of naturally or spontaneously induced immunity can be observed. This might be the demonstration of antigen-specific CD8 or CD4 T cells in a high frequency of cancer patients with antigen expressing tumors or the presence of autologous antigen-specific antibodies in such cancer patients, preferably both (Jager et al. PNAS 2000 97:4700-5; Gnjatic et al. PNAS 2003 100:8862-7).

The invention involves the use of various materials disclosed herein to “immunize” subjects or as “vaccines”. As used herein, “immunization” or “vaccination” means increasing or activating an immune response against an antigen. It does not require elimination or eradication of a condition but rather contemplates the clinically favorable enhancement of an immune response toward an antigen. Generally accepted animal models can be used for testing of immunization against cancer using one or more MRC markers. For example, human cancer MRCs can be introduced into a mouse to create a tumor, and one or more MRC markers (or nucleic acids encoding these) can be delivered by the methods described herein. The effect on the cancer cells (e.g., reduction of tumor size) can be assessed as a measure of the effectiveness of the MRC marker nucleic acid immunization. Of course, testing of the foregoing animal model using more conventional methods for immunization can include the administration of one or more MRC marker polypeptides or fragments derived therefrom, optionally combined with one or more adjuvants and/or cytokines to boost the immune response.

Methods for immunization, including formulation of a vaccine composition and selection of doses, route of administration and the schedule of administration (e.g. primary and one or more booster doses), are well known in the art. The tests also can be performed in humans, where the end point is to test for the presence of enhanced levels of circulating CTLs against MRCs, to test for levels of circulating antibodies against the one or more MRC markers, to test for the presence of cells expressing the antigen and so forth.

As part of the immunization compositions, one or more MRC markers or immunogenic fragments thereof are administered with one or more adjuvants to induce an immune response or to increase an immune response. An adjuvant is a substance incorporated into or administered with antigen which potentiates the immune response. Adjuvants may enhance the immunological response by providing a reservoir of antigen (extracellularly or within macrophages), activating macrophages and stimulating specific sets of lymphocytes. Adjuvants of many kinds are well known in the art. Specific examples of adjuvants include monophosphoryl lipid A (MPL, SmithKline Beecham), a congener obtained after purification and acid hydrolysis of Salmonella minnesota Re 595 lipopolysaccharide; saponins including QS21 (SmithKline Beecham), a pure QA-21 saponin purified from Quillja saponaria extract; DQS21, described in PCT application WO96/33739 (SmithKline Beecham), ISCOMATRIX® (CSL Ltd., Parkville, Victoria, Australia) derived from the bark of the Quillaia saponaria molina tree; QS-7, QS-17, QS-18, and QS-L1 (So et al., Mol. Cells. 7:178-186, 1997); incomplete Freund's adjuvant; complete Freund's adjuvant; montanide; alum; CpG oligonucleotides (see e.g. Kreig et al., Nature 374:546-9, 1995; U.S. Pat. No. 6,207,646) and other immunostimulatory oligonucleotides; various water-in-oil emulsions prepared from biodegradable oils such as squalene and/or tocopherol; and factors that are taken up by the so-called ‘toll-like receptor 7’ on certain immune cells that are found in the outside part of the skin, such as imiquimod (3M, St. Paul, Minn.). Preferably, the antigens are administered mixed with a combination of DQS21/MPL or ISCOMATRIX®. The ratio of DQS21 to MPL typically will be about 1:10 to 10:1, preferably about 1:5 to 5:1 and more preferably about 1:1. Typically for human administration, DQS21 and MPL will be present in a vaccine formulation in the range of about 1 μg to about 100 μg. Other adjuvants are known in the art and can be used in the invention (see, e.g. Goding, Monoclonal Antibodies: Principles and Practice, 2nd Ed., 1986). Methods for the preparation of mixtures or emulsions of polypeptide and adjuvant are well known to those of skill in the art of vaccination.

Other agents which stimulate the immune response of the subject can also be administered to the subject. For example, other cytokines are also useful in vaccination protocols as a result of their lymphocyte regulatory properties. Many other cytokines useful for such purposes will be known to one of ordinary skill in the art, including interleukin-12 (IL-12) which has been shown to enhance the protective effects of vaccines (see, e.g., Science 268: 1432-1434, 1995), GM-CSF, IL-18 and IL-15 (Klebanoff et al. Proc. Natl. Acad. Sci. USA 2004 101:1969-74). Thus cytokines can be administered in conjunction with antigens and adjuvants to increase the immune response to the antigens.

There are a number of immune response potentiating compounds that can be used in vaccination protocols. These include costimulatory molecules provided in either protein or nucleic acid form. Such costimulatory molecules include the B7-1 and B7-2 (CD80 and CD86 respectively) molecules which are expressed on dendritic cells (DC) and interact with the CD28 molecule expressed on the T cell. This interaction provides costimulation (signal 2) to an antigen/MHC/TCR stimulated (signal 1) T cell, increasing T cell proliferation and effector function. B7 also interacts with CTLA4 (CD152) on T cells and studies involving CTLA4 and B7 ligands indicate that the B7-CTLA4 interaction can enhance antitumor immunity and CTL proliferation (Zheng P., et al. Proc. Natl. Acad. Sci. USA 95 (11):6284-6289 (1998)).

B7 typically is not expressed on tumor cells so they are not efficient antigen presenting cells (APCs) for T cells. Induction of B7 expression would enable the tumor cells to stimulate more efficiently CTL proliferation and effector function. A combination of B7/IL-6/IL-12 costimulation has been shown to induce IFN-gamma and a Th1 cytokine profile in the T cell population leading to further enhanced T cell activity (Gajewski et al., J. Immunol, 154:5637-5648 (1995)). Tumor cell transfection with B7 has been discussed in relation to in vitro CTL expansion for adoptive transfer immunotherapy by Wang et al., (J. Immunol., 19:1-8 (1986)). Other delivery mechanisms for the B7 molecule include nucleic acid (naked DNA) immunization (Kim J., et al. Nat. Biotechnol., 15:7:641-646 (1997)) and recombinant viruses such as adeno and pox (Wendtner et al., Gene Ther., 4:7:726-735 (1997)). These systems are all amenable to the construction and use of expression cassettes for the coexpression of B7 with other molecules of choice such as the antigens or fragment(s) of antigens discussed herein (including polytopes) or cytokines. These delivery systems can be used for induction of the appropriate molecules in vitro and for in vivo vaccination situations. The use of anti-CD28 antibodies to directly stimulate T cells in vitro and in vivo could also be considered. Similarly, the inducible co-stimulatory molecule ICOS which induces T cell responses to foreign antigen could be modulated, for example, by use of anti-ICOS antibodies (Hutloff et al., Nature 397:263-266, 1999).

Lymphocyte function associated antigen-3 (LFA-3) is expressed on APCs and some tumor cells and interacts with CD2 expressed on T cells. This interaction induces T cell IL-2 and IFN-gamma production and can thus complement but not substitute, the B7/CD28 costimulatory interaction (Parra et al., J. Immunol., 158:637-642 (1997), Fenton et al., J. Immunother., 21:2:95-108 (1998)).

Lymphocyte function associated antigen-1 (LFA-1) is expressed on leukocytes and interacts with ICAM-1 expressed on APCs and some tumor cells. This interaction induces T cell IL-2 and IFN-gamma production and can thus complement but not substitute, the B7/CD28 costimulatory interaction (Fenton et al., J. Immunother., 21:2:95-108 (1998)). LFA-1 is thus a further example of a costimulatory molecule that could be provided in a vaccination protocol in the various ways discussed above for B7.

Complete CTL activation and effector function requires Th cell help through the interaction between the Th cell CD40L (CD40 ligand) molecule and the CD40 molecule expressed by DCs (Ridge et al., Nature, 393:474 (1998), Bennett et al., Nature, 393:478 (1998), Schoenberger et al., Nature, 393:480 (1998)). This mechanism of this costimulatory signal is likely to involve upregulation of B7 and associated IL-6/IL-12 production by the DC (APC). The CD40-CD40L interaction thus complements the signal 1 (antigen/MHC-TCR) and signal 2 (B7-CD28) interactions.

The use of anti-CD40 antibodies to stimulate DC cells directly, would be expected to enhance a response to tumor antigens which are normally encountered outside of an inflammatory context or are presented by non-professional APCs (tumor cells). In these situations Th help and B7 costimulation signals are not provided.

The invention contemplates delivery of nucleic acids, polypeptides or fragments thereof for vaccination. Delivery of polypeptides and fragments thereof can be accomplished according to standard vaccination protocols which are well known in the art. In another embodiment, the delivery of nucleic acid is accomplished by ex vivo methods, i.e. by removing a cell from a subject, genetically engineering the cell to include one or more MRC markers, and reintroducing the engineered cell into the subject. One example of such a procedure is outlined in U.S. Pat. No. 5,399,346 and in exhibits submitted in the file history of that patent, all of which are publicly available documents. In general, it involves introduction in vitro of a functional copy of a gene into a cell(s) of a subject, and returning the genetically engineered cell(s) to the subject. The functional copy of the gene is under operable control of regulatory elements which permit expression of the gene in the genetically engineered cell(s). Numerous transfection and transduction techniques as well as appropriate expression vectors are well known to those of ordinary skill in the art, some of which are described in PCT application WO95/00654. In vivo nucleic acid delivery using vectors such as viruses and targeted liposomes also is contemplated according to the invention.

A virus vector for delivering a nucleic acid encoding one or more MRC markers is selected from the group consisting of adenoviruses, adeno-associated viruses, poxviruses including vaccinia viruses and attenuated poxviruses, Semliki Forest virus, Venezuelan equine encephalitis virus, retroviruses, Sindbis virus, and Ty virus-like particle. Examples of viruses and virus-like particles which have been used to deliver exogenous nucleic acids include: replication-defective adenoviruses (e.g., Xiang et al., Virology 219:220-227, 1996; Eloit et al., J. Virol. 7:5375-5381, 1997; Chengalvala et al., Vaccine 15:335-339, 1997), a modified retrovirus (Townsend et al., J. Virol. 71:3365-3374, 1997), a nonreplicating retrovirus (Irwin et al., J. Virol. 68:5036-5044, 1994), a replication defective Semliki Forest virus (Zhao et al., Proc. Natl. Acad. Sci. USA 92:3009-3013, 1995), canarypox virus and highly attenuated vaccinia virus derivative (Paoletti, Proc. Natl. Acad. Sci. USA 93:11349-11353, 1996), non-replicative vaccinia virus (Moss, Proc. Natl. Acad. Sci. USA 93:11341-11348, 1996), replicative vaccinia virus (Moss, Dev. Biol. Stand. 82:55-63, 1994), Venzuelan equine encephalitis virus (Davis et al., J. Virol. 70:3781-3787, 1996), Sindbis virus (Pugachev et al., Virology 212:587-594, 1995), and Ty virus-like particle (Allsopp et al., Eur. J. Immunol. 26:1951-1959, 1996). A preferred virus vector is an adenovirus.

Preferably the foregoing nucleic acid delivery vectors: (1) contain exogenous genetic material that can be transcribed and translated in a mammalian cell and that can induce an immune response in a host, and (2) contain on a surface a ligand that selectively binds to a receptor on the surface of a target cell, such as a mammalian cell, and thereby gains entry to the target cell.

Various techniques may be employed for introducing nucleic acids into cells, depending on whether the nucleic acids are introduced in vitro or in vivo in a host. Such techniques include transfection of nucleic acid-CaPO₄ precipitates, transfection of nucleic acids associated with DEAE, transfection or infection with the foregoing viruses including the nucleic acid of interest, liposome mediated transfection, and the like. For certain uses, it is preferred to target the nucleic acid to particular cells. In such instances, a vehicle used for delivering a nucleic acid into a cell (e.g., a retrovirus, or other virus; a liposome) can have a targeting molecule attached thereto. For example, a molecule such as an antibody specific for a surface membrane protein on the target cell or a ligand for a receptor on the target cell can be bound to or incorporated within the nucleic acid delivery vehicle. Preferred antibodies include antibodies which selectively bind a cancer-testis antigen, alone or as a complex with a MHC molecule. Especially preferred are monoclonal antibodies. Where liposomes are employed to deliver the nucleic acids, proteins which bind to a surface membrane protein associated with endocytosis may be incorporated into the liposome formulation for targeting and/or to facilitate uptake. Such proteins include capsid proteins or fragments thereof tropic for a particular cell type, antibodies for proteins which undergo internalization in cycling, proteins that target intracellular localization and enhance intracellular half life, and the like. Polymeric delivery systems also have been used successfully to deliver nucleic acids into cells, as is known by those skilled in the art. Such systems even permit oral delivery of nucleic acids.

According to a further aspect of the invention, compositions containing the nucleic acid molecules, proteins, and binding polypeptides are provided. The compositions contain any of the foregoing nucleic acid molecules, proteins, and binding polypeptides (as therapeutic agents) in an optional pharmaceutically acceptable carrier. Thus, in a related aspect, the invention provides a method for forming a medicament that involves placing a therapeutically effective amount of the therapeutic agent in the pharmaceutically acceptable carrier to form one or more doses. The effectiveness of treatment or prevention methods of the invention can be determined using standard diagnostic methods described herein.

When administered, the therapeutic compositions of the present invention are administered in pharmaceutically acceptable preparations. Such preparations may routinely contain pharmaceutically acceptable concentrations of salt, buffering agents, preservatives, compatible carriers, supplementary immune potentiating agents such as adjuvants and cytokines, and optionally other therapeutic agents.

As used herein, the term “pharmaceutically acceptable” means a non-toxic material that does not interfere with the effectiveness of the biological activity of the active ingredients. The term “physiologically acceptable” refers to a non-toxic material that is compatible with a biological system such as a cell, cell culture, tissue, or organism. The characteristics of the carrier will depend on the route of administration. Physiologically and pharmaceutically acceptable carriers include diluents, fillers, salts, buffers, stabilizers, solubilizers, and other materials which are well known in the art. The term “carrier” denotes an organic or inorganic ingredient, natural or synthetic, with which the active ingredient is combined to facilitate the application. The components of the pharmaceutical compositions also are capable of being co-mingled with the molecules of the present invention, and with each other, in a manner such that there is no interaction which would substantially impair the desired pharmaceutical efficacy.

The therapeutics of the invention can be administered by any conventional route, including injection or by gradual infusion over time. The administration may, for example, be oral, intravenous, intratumoral, intraperitoneal, intramuscular, intracavity, subcutaneous, or transdermal. When antibodies are used therapeutically, a preferred route of administration is by pulmonary aerosol. Techniques for preparing aerosol delivery systems containing antibodies are well known to those of skill in the art. Generally, such systems should utilize components which will not significantly impair the biological properties of the antibodies, such as the paratope binding capacity (see, for example, Sciarra and Cutie, “Aerosols,” in Remington's Pharmaceutical Sciences, 18th edition, 1990, pp 1694-1712). Those of skill in the art can readily determine the various parameters and conditions for producing antibody aerosols without undue experimentation. When using antisense preparations of the invention, slow intravenous administration is preferred.

The compositions of the invention are administered in effective amounts. An “effective amount” is that amount of a composition that alone, or together with further doses, produces the desired response, e.g. increases an immune response to the one or more MRC markers. In the case of treating a particular disease or condition characterized by the presence of MRCs, such as cancer, the desired response is inhibiting the progression of the disease. This may involve only slowing the progression of the disease temporarily, although more preferably, it involves halting the progression of the disease permanently. This can be monitored by routine methods or can be monitored according to diagnostic methods of the invention discussed herein. The desired response to treatment of the disease or condition also can be delaying the onset or even preventing the onset of the disease or condition.

Such amounts will depend, of course, on the particular condition being treated, the severity of the condition, the individual patient parameters including age, physical condition, size and weight, the duration of the treatment, the nature of concurrent therapy (if any), the specific route of administration and like factors within the knowledge and expertise of the health practitioner. These factors are well known to those of ordinary skill in the art and can be addressed with no more than routine experimentation. It is generally preferred that a maximum dose of the individual components or combinations thereof be used, that is, the highest safe dose according to sound medical judgment. It will be understood by those of ordinary skill in the art, however, that a patient may insist upon a lower dose or tolerable dose for medical reasons, psychological reasons or for virtually any other reasons.

The pharmaceutical compositions used in the foregoing methods preferably are sterile and contain an effective amount of one or more MRC marker polypeptides or nucleic acids encoding one or more MRC markers for producing the desired response in a unit of weight or volume suitable for administration to a patient. The response can, for example, be measured by determining the immune response following administration of the MRC marker composition via a reporter system by measuring downstream effects such as gene expression, or by measuring the physiological effects of the MRC marker polypeptide composition, such as regression of a tumor, decrease of MRCs or decrease of disease symptoms. Other assays will be known to one of ordinary skill in the art and can be employed for measuring the level of the response.

The doses of one or more MRC marker compositions (e.g., polypeptide, peptide, antibody, cell or nucleic acid) administered to a subject can be chosen in accordance with different parameters, in particular in accordance with the mode of administration used and the state of the subject. Other factors include the desired period of treatment. In the event that a response in a subject is insufficient at the initial doses applied, higher doses (or effectively higher doses by a different, more localized delivery route) may be employed to the extent that patient tolerance permits.

In general, for treatments for eliciting or increasing an immune response, doses of one or more MRC markers are formulated and administered in doses between 1 ng and 1 mg, and preferably between 10 ng and 100 μg, according to any standard procedure in the art. Where nucleic acids encoding one or more MRC markers or variants thereof are employed, doses of between 1 ng and 0.1 mg generally will be formulated and administered according to standard procedures. Other protocols for the administration of one or more MRC marker compositions will be known to one of ordinary skill in the art, in which the dose amount, schedule of injections, sites of injections, mode of administration (e.g., intra-tumoral) and the like vary from the foregoing. Administration of MRC marker polypeptide compositions to mammals other than humans, e.g. for testing purposes or veterinary therapeutic purposes, is carried out under substantially the same conditions as described above.

Where one or more MRC marker polypeptides are used for vaccination, modes of administration which effectively deliver the MRC marker polypeptide and adjuvant, such that an immune response to the polypeptide is increased, can be used. For administration of a MRC marker polypeptide in adjuvant, preferred methods include intradermal, intravenous, intramuscular and subcutaneous administration. Although these are preferred embodiments, the invention is not limited by the particular modes of administration disclosed herein. Standard references in the art (e.g., Remington's Pharmaceutical Sciences, 18th edition, 1990) provide modes of administration and formulations for delivery of immunogens with adjuvant or in a non-adjuvant carrier.

The pharmaceutical compositions may contain suitable buffering agents, including: acetic acid in a salt; citric acid in a salt; boric acid in a salt; and phosphoric acid in a salt.

The pharmaceutical compositions also may contain, optionally, suitable preservatives, such as: benzalkonium chloride; chlorobutanol; parabens and thimerosal.

The pharmaceutical compositions may conveniently be presented in unit dosage form and may be prepared by any of the methods well-known in the art of pharmacy. All methods include the step of bringing the active agent into association with a carrier which constitutes one or more accessory ingredients. In general, the compositions are prepared by uniformly and intimately bringing the active compound into association with a liquid carrier, a finely divided solid carrier, or both, and then, if necessary, shaping the product.

Compositions suitable for oral administration may be presented as discrete units, such as capsules, tablets, lozenges, each containing a predetermined amount of the active compound. Other compositions include suspensions in aqueous liquids or non-aqueous liquids such as a syrup, elixir or an emulsion.

Compositions for parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, and lactated Ringer's or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases, and the like.

The pharmaceutical agents of the invention may be administered alone, in combination with each other, and/or in combination with other anti-cancer drug therapies and/or treatments. These therapies and/or treatments may include, but are not limited to: surgical intervention, chemotherapy, radiotherapy, and adjuvant systemic therapies.

The invention also provides a pharmaceutical kit comprising one or more containers comprising one or more of the pharmaceutical compounds or agents of the invention. Additional materials may be included in any or all kits of the invention, and such materials may include, but are not limited to buffers, water, enzymes, tubes, control molecules, etc. The kit may also include instructions for the use of the one or more pharmaceutical compounds or agents of the invention for the treatment of cancer.

### EXAMPLES

Melanoma is increasing in incidence in Western countries, and accounts for more years of life lost than any other adult malignancy. Patients with metastatic disease exhibit variable clinical courses, but treatments such as chemotherapy and radiotherapy are largely ineffective. Observed spontaneous immune responses to melanoma have led to the pursuit of immunotherapeutic options for melanoma treatment. Recent successes include antibodies to CTLA-4⁴², and adoptive immunotherapy with ex vivo expanded MART-1 specific CD8 T-cells⁴³. Despite a paucity of objective responses, much work continues to focus on active vaccination, which is influenced by factors such as the stage of disease, the accompanying adjuvant, the treatment schedule and the target antigen of choice.

The cancer/testis antigens (CTAg) are highly suitable antigens for cancer immunotherapy¹⁴. Originally discovered by SEREX screening of cancer patients' serum the CTAg are expressed in germ cells of the testis, ovary and trophoblast¹³, and re-expressed in a wide variety of tumours, with considerable inter- and intra-tumoral variability. Though their functions remain unknown there is speculation that they contribute to of germ cell functions such as self-renewal. MAGE-Al has been found to be a transcriptional co-repressor⁴⁴, and the CTAg may have integrated functions as CT7 has been shown to physically interact with NY-ESO-1⁴⁵.

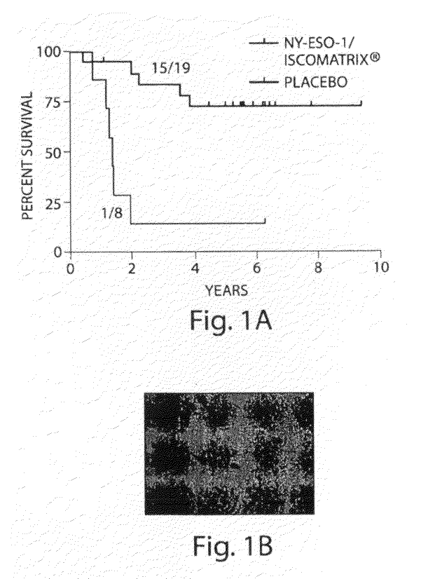

We have previously reported³¹ evidence of a broad humoral and cellular immunological response generated by vaccination of patients with resected stage III/IV melanoma with the combination of the full-length protein of the cancer/testis antigen NY-ESO-1 in combination with the potent adjuvant ISCOMATRIX®. In an unplanned analysis of this randomized phase I/II trial, patients who received the vaccine appear to have improved relapse free survival compared to patients who received placebo (FIG. 1a). This result was intriguing as many of the vaccinated patients had tumors where only a minority of cells expressed NY-ESO-1 (a representative immunohistochemistry section is shown; FIG. 1b). In fact more than half of the patients in the active vaccine group had tumors in which 50% or less of the tumor cells were positive for NY-ESO-1 by immunohistochemistry. Though this apparent survival advantage may simply represent a type I error in a small trial, a review of the patient characteristics showed no obvious differences in known prognostic factors. An attractive alternative hypothesis is that the broad immune response generated by the NY-ESO-1/ISCOMATRLX® vaccine specifically targeted the critical cells within the melanoma responsible for tumorigenic potential. We hypothesize that the vaccine had stimulated an immune response that directly targeted melanoma stem cells.

The cancer stem cell hypothesis⁴⁶ posits that a hierarchy exists within each cancer with only a subpopulation of cells being capable of self-renewal, extensive proliferation and new tumor formation. Cancer stem cells have been identified for an increasing number of malignancies including acute myeloid leukemia, breast carcinoma, prostate carcinoma, head and neck cancer and colorectal cancer⁴⁷. The CSC hypothesis also accounts for many clinical cancer observations including intra-tumoral differentiation³, cancer cell migration⁶ and intrinsic cytotoxic resistance resulting in treatment failure⁷. Based on the finding that CD133 marks a stem cell population in glioblastoma and medulloblastoma⁴⁸, and a skin-resident neural crest stem cell population⁴⁹, we sought to investigate whether CD133 identified a cancer stem cell population in melanoma, a cancer also presumably deriving from the neural crest lineage.

### Example 1

### Identification of Melanoma Repopulating Cells and Phenotypic Characterization

We studied the pattern of NY-ESO-1 distribution in primary and metastatic melanomas. Unlike the differentiation antigens (Ag) which were widely expressed in the majority of tumour cells, cancer-testis antigens (CTAg) were only expressed in a subset of tumours irrespective of stage. Furthermore most Ag+ cells only displayed CTAg expression in a subset of cells, frequently <25% of cells.

### Evaluation of CD133-Specific Antibodies

Antibodies against CD133 have different specificities. Differences can be attributed to whether or not they detect a splice variant known as CD133-2³² which has an 8 by deletion near the 5′ end and variable glycosylation of CD133³³. We compared the following commercially available antibodies: (i) AC133: mouse monoclonal IgG1 (Miltenyi), (ii) AC141 (Miltenyi) mouse monoclonal IgG1, (iii) 293C3: mouse monoclonal IgG2b (Miltenyi), and (iv) a polyclonal antiserum, rabbit polyclonal ab 16518 (Abeam). These were tested on melanoma cell lines which had been cultured on tissue culture slides: LAR1a, LAR14, LAR17 and SK-MEL 24 and SK-MEL 25. Staining of a subset of cells (1-3%) was seen in 4 of 5 lines. The yield of CD133hi cells was highest for AC141 and 293C3 (Table 1).

TABLE 1

Antibody

| Cell line | AC133 | AC141 | 293C3 | 16518 |
|---|---|---|---|---|
| CD133 variant | 133-1 | 133-2 | 133-2 | |
| LAR1a | | + | + | |
| LAR14 | | | | |
| LAR17 | | + | + | |
| SK-MEL 24 | + | + | + | |
| LAR49 | | + | + | |

### Melanoma Tissue Sections and Melanoma Cell Lines

To confirm that CD133 expression occurred in tumours and was not just a feature of cell lines, expression of CD133 was evaluated with these antibodies in frozen and paraffin embedded tissue sections using cell blocks and melanoma tissue sections by immunohistochemistry (IHC). Clusters of CD133hi cells were seen in all tissues tested and there was good concordance with cell lines derived from these tumours.

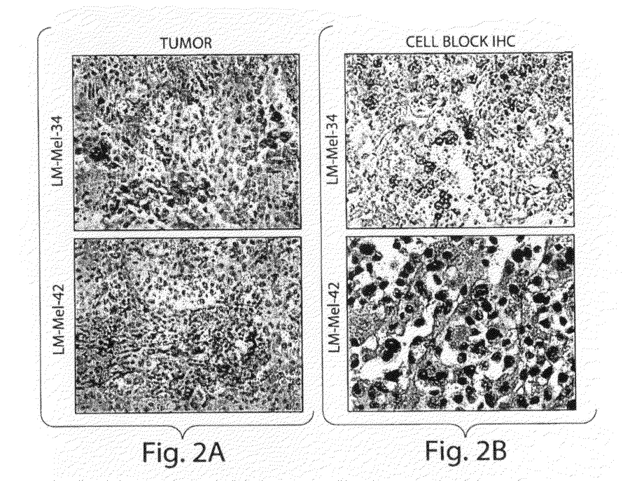

Immunohistochemistry of frozen melanoma tissue specimens revealed 9 of 23 tumours, and 6 of 32 melanoma cell lines surveyed contained CD133⁺ cells (for examples see FIGS. 2a, b). Typically isolated single cells or small clusters stained positive, but occasional tumours and cell lines contained many CD133⁺ cells. Staining with antibodies to the CD133-2 epitope identified a higher percentage of expression of CD133 in cell lines, compared to the AC133 and AC141 antibodies. Immunohistochemistry performed using the pure AC133 antibody and a rabbit polyclonal anti-CD133 antibody (Abcam, Cambridge UK) showed extensive non-specific and background staining (data not shown). CD133 expression appears to be a continuum in melanoma cell lines, with the majority of cells staining negative (CD133^{neg}), and a penumbra of cells showing higher expression (CD133⁴).

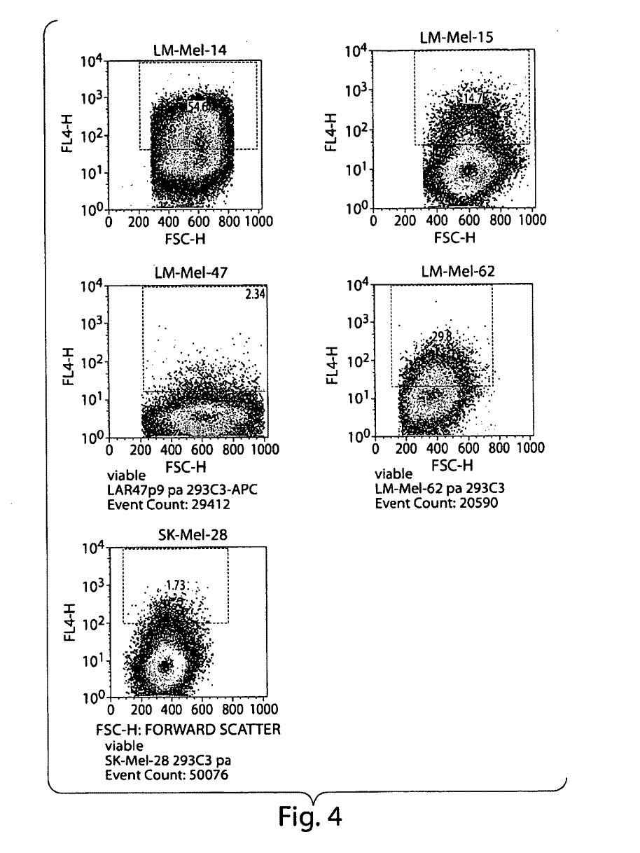

Occasional early passage cell lines had high expression at early passage (FIG. 4), whilst higher-passage cell lines such as SK-MeI-28 also had a high proportion of CD133⁺ cells.

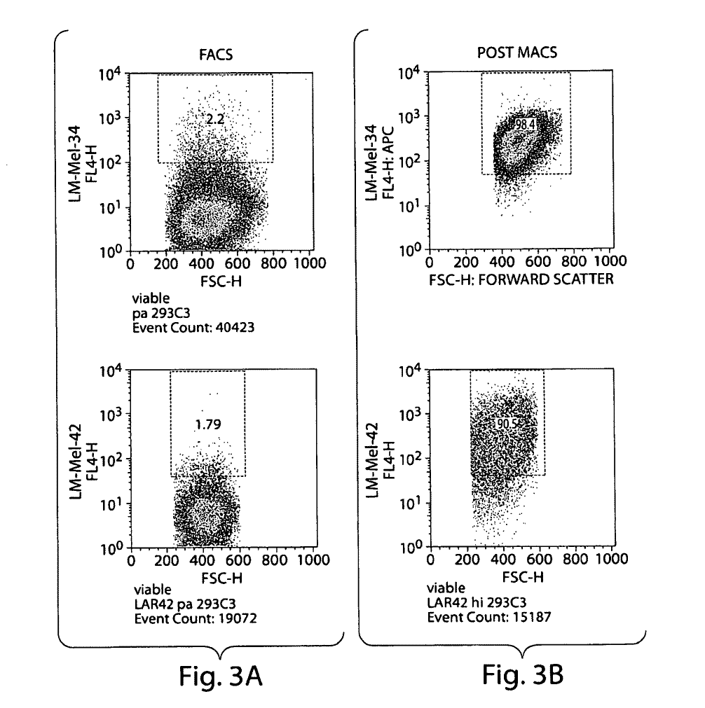

After enrichment by MACS and flow cytometry sorting, CD133⁺ cells typically represented 90-95% of the cell population, whereas CD133^{neg} cells were depleted to <0.5% (FIGS. 3a, b).

### Growth Characteristics of CD133hi Cells

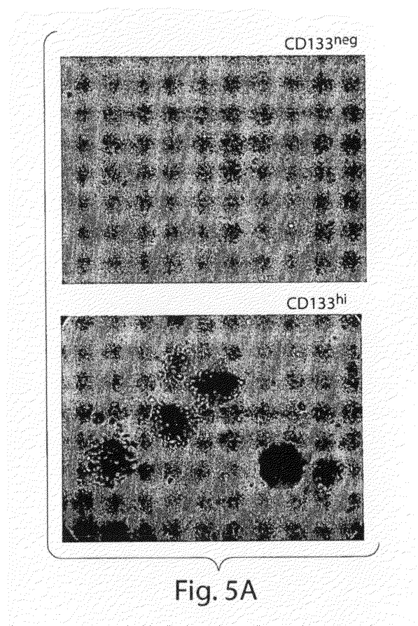

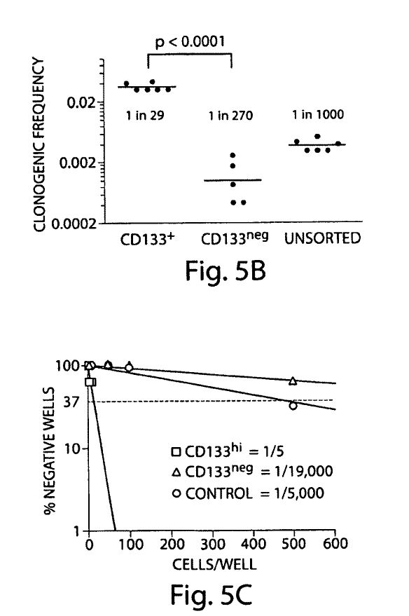

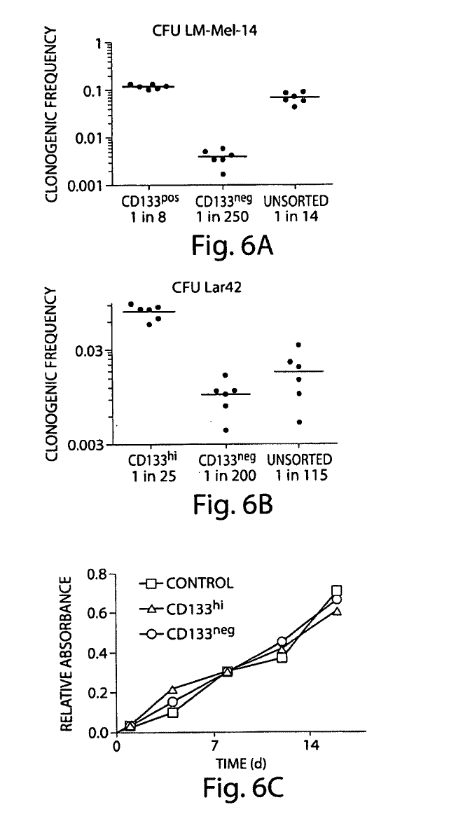

CD133hi melanoma cells were studied in assays of soft agar colony formation assay (FIGS. 5a, b, c) and proliferation (FIG. 6c).

To assess the clonogenicity of CD133⁺ melanoma cells we employed the colony forming unit assay in soft agar. CD133⁺ cells were highly enriched for clonogenic potential compared to CD133^{neg} or unsorted control cells (FIGS. 5a, b). This finding could be replicated in several melanoma cell lines (FIGS. 6a, b; LM-MeI-14 and LAR42, respectively) and when cells were grown at limiting dilution. When grown at limiting dilution, CD133hi cells were more likely to form new colonies (FIG. 5c); In contrast CD133⁺ cells are no more proliferative in short-term proliferation assays compared to CD133^{neg} and control cells (FIG. 6c).

### CD133hi Melanoma Cells Show Evidence of In Vitro Self-Renewal and Differentiation.

When CD133 separated cells were returned to bulk culture, and reanalyzed by flow cytometry several weeks later, CD133⁺ cells give rise to CD133^{neg} cells, but CD133^{neg} cells were unable to form CD133⁺ cells, indicating a hierarchical relationship (FIGS. 7a, b, d). Enriched CD133⁺ melanoma cells were sorted into single cells in 96-well plates, without feeder cells or conditioned medium. A proportion of CD133⁺ cells proliferated extensively, and could be passaged from a single cell to >10⁷ cells. Cultures derived from these single cells had biphasic expression of CD133, and CD133⁺ clones could be serial sorted in further rounds of single-cell sorting, with subsequent cell colonies being derived from a single CD133⁺ cell. In contrast CD133^{neg} cells could occasionally proliferate, but most often formed diffuse of cells with more complex and heterogeneous morphology (FIG. 7c). These populations remained negative for CD133 expression, and subsequent single sorted CD133^{neg} cells from these populations did not proliferate. Conversely, isolated CD133⁺ derived colonies grown in agar were plucked under direct visualization, then cultured in liquid medium, showing extensive proliferation. Cell populations derived from these colonies again showed heterogeneous CD133 expression, and when enriched and depleted, once again showed that the clonogenic potential resided in the CD133⁺ fraction (data not shown).

CTAg are Predominantly Expressed in CD133hi cells

To investigate CTAg expression in CD133⁺ clonogenic melanoma cells we performed immunohistochemistry and quantitative real-time PCR on CD133 enriched and depleted cell block preparations. Sorted cells were evaluated for CTAg expression by IHC and RT-PCR. A panel of antigens were assessed: CD133, NY-ESO-1, MAGEA3/A4, MAGEA1, CT7 and MelanA, which served as a non-CTAg control.

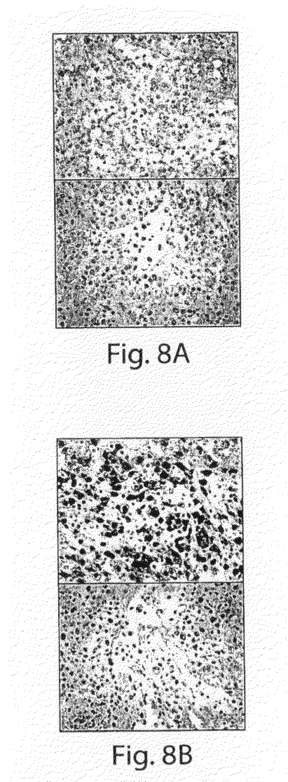

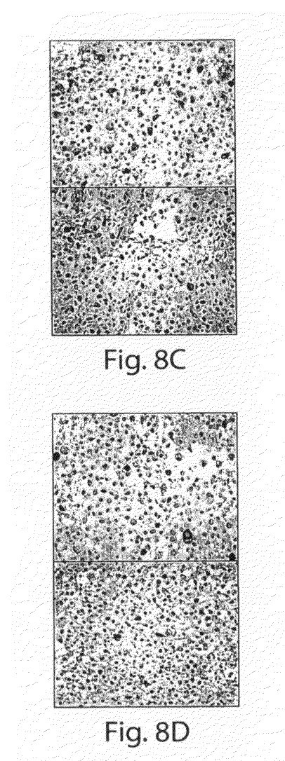

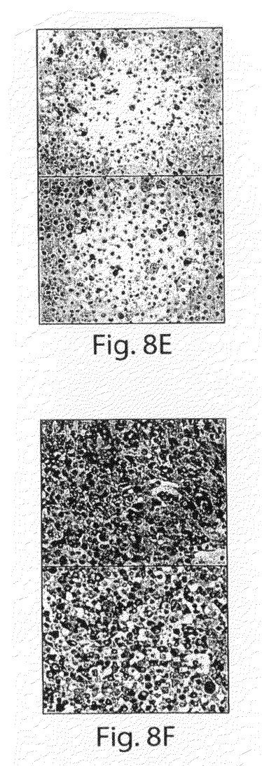

FIG. 8a-f shows LAR34 melanoma cells that were MACS separated into CD133hi (top panels) and CD133neg (bottom panels). The panels show immunohistochemistry of cell blocks (200×), as follows: FIG. 8a) CD133, FIG. 8b) NY-ESO-1, FIG. 8c) polyMAGE (MAGEA3/A4), FIG. 8d) MAGEC1, FIG. 8e) MAGEA1, and FIG. 8f) Melan-A.

CD133⁺ melanoma stem-like cells show enriched expression for NY-ESO-1 and MAGEA4 compared to CD133^{neg} cells (FIGS. 8b, c).

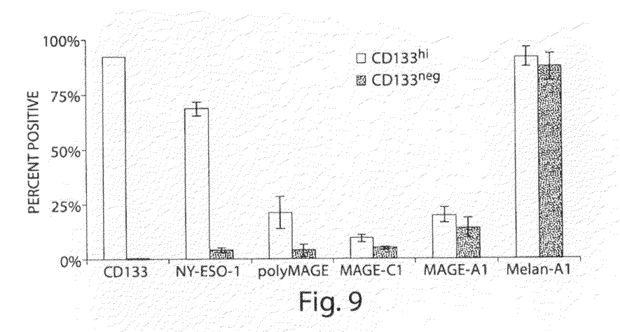

FIG. 9 shows that in cell counts from randomly selected high-power fields of each cell block (mean±SD) all CTAg expression segregated into the CD133hi cell fraction. This was most marked for NY-ESO-1 in which case virtually none of the CD133neg cells expressed this CTAg. Expression of other CTAg and differentiation antigens was similar.

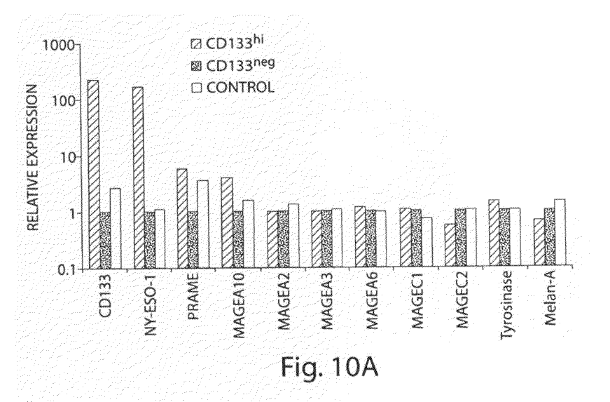

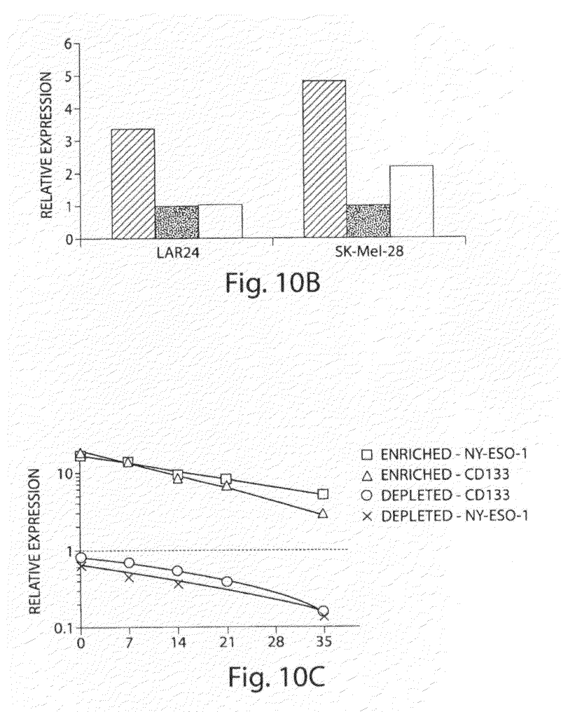

CTAg expression was also assessed by real-time PCR. LAR34CD133hi cells demonstrated higher expression of the CTAg NY-ESO-1, MAGEA10 and PRAME in the CD133⁺ population (FIG. 10a). Expression of other CT and differentiation antigens tested was not significantly different. CTAg were more highly expressed in CD133⁺ clonogenic cells in other melanoma cell lines (data not shown). LAR24 and SK-MeI-28 CD133hi melanoma cell lines also demonstrated high expression of NY-ESO-1 compared to CD133neg and control cells (FIG. 10b).

CD133⁺ melanoma cells were returned to culture, and passaged at intervals. CTAg expression in the resulting populations was examined by RT-PCR (FIG. 10c). Expression of CD133 decayed over time taking several weeks to return to return to the same level as unsorted cells, while NY-ESO-1 expression also declined but less rapidly. The CD133^{neg} population showed minimal declines in expression of both CD133 and NY-ESO-1.

### Example 2

### Further Characterization of Melanoma Repopulating Cells

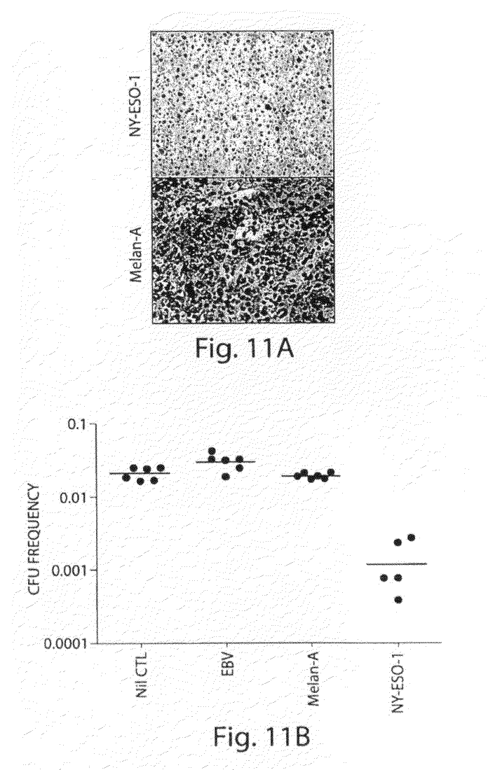

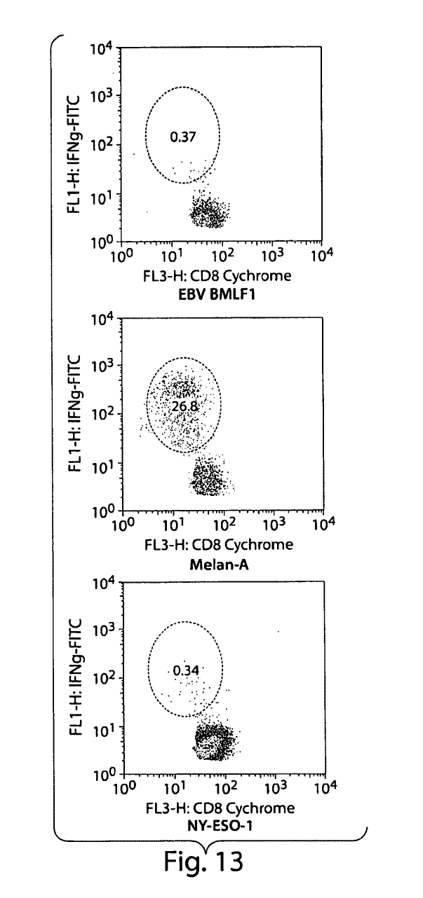

Further molecular characterization of MRC is undertaken for two purposes (i) to identify potential additional cell surface markers that may enable further purification of MRC from within the CD133hi subset and (ii) to characterize Ag expression for the purpose of immune targeting. CTAg are proven targets for immune recognition and represent the first known tumour-specific targets known to be selectively expressed on MRC. Purified cell populations therefore are characterized for these molecules, extending the results of the studies reported herein (FIG. 11a,b and FIG. 13).

### Confirmation of Results Using Tumours and Cell Lines

As shown herein, CD133 has proven to be an extremely helpful marker for clonogenic melanoma cells in vitro. These data are confirmed by undertaking studies on additional cell lines and tumours. This is done to establish whether the observations made to date are more widely applicable. Fifty melanoma cell lines derived from patients of the Ludwig Institute Melanoma program at Austin Health are studied. For these lines comparisons with the harvested tumours (stored as both fresh frozen blocks in OCT and embedded in paraffin) are performed for CD133 expression as well as for the expression of CTAg. Validated antibodies for IHC are available against MAGEA1, -A2, -A3, -A4, NY-ESO-1, CT7, CT10 and SSX-1. Furthermore mRNA has previously been prepared for RT-PCR-based tumour Ag typing so expression of additional CTAg, particularly those for which no antibodies are available can be performed. Following the screening of available lines by IHC and RT-PCR, representative lines will be selected for further study.

### Identification of Additional Cell Surface Markers

As shown herein, a clearly definable subset of CD133hi cells appear to be MRC (as exemplified by LAR24, LAR34 and SK-MEL 28). Initial experiments have shown the frequency of clonogenic cells within the CD133+ compartment can vary from as high as ⅕ (LAR34) to 1/75 (SK-MEL 28). To further characterise the clonogenic population, additional markers are identified. A series of candidate molecules is tested empirically by screening cells sorted on the basis of CD133. These are cell surface markers which have previously been described on melanocytes and their precursors MUC 18 (CD146), M10, M2, M3³⁶), including neural lineage stem-cell markers such as nestin, PSA-NCAM, α-tubulin type III and microtubule-associated protein 2 (MAP2)³⁷ and markers for stem cells such as the SSEAs, ABCG2 and Sca-1³⁸. For the cell-surface markers that are found to label a subset within the CD133 compartment, cells are sorted using immuno-magnetic beads or by automated cell sorting, and are evaluated for clonogenicity in the agar colony assay. Antibodies to candidate molecules are selected and used to sort labelled cells and then tested in the colony assay.

For all studies described below, analyses are initially undertaken using cells sorted on the basis of CD133 expression. As shown above, this has been sufficient to identify the presence of MRC and notably the segregation of CTAg expression into the CD133hi compartment. We therefore have shown that these cells are sufficient to perform the following experiments. These studies are performed with the more highly purified cells in the event that an additional marker helps to define a purer clonogenic subset.

### Characterization of CTAg Expression in MRC:

IHC and dual staining by immunofluorescence establish the extent to which CD133 hi and lo cells express each of NY-ESO-1, MAGEA1, A3, A4, CT 7, CT10, and other CTAg for which antibodies are available. Quantitative RT PCR is performed for these and other CTAg for which antibodies are not available. PCR primers currently available within our laboratory are listed on Table 2 (and are exemplified in Table 3):

TABLE 2

| | |
|---|---|
| Melanoma | CD133/Prominin-1 |
| Stem/ | ABCB5 |
| Progenitor | NY-ESO-1/CT6.1(and Real Time PCR) |
| genes | LAGE-1a/CT6.2a (and Real Time PCR) |
| | MAGEA1/CT1.1 |
| | MAGEA3/CT1.3 |
| | MAGEA4/CT1.4 (*4 transcript variants) |
| | MAGEC1 (CT7) |
| | MAGEA10/CT1.10 |
| | CT10/MAGEE1 |
| | MAGEA6/CT1.6 |
| | SSX-1/CT5.1 |
| | SCP-1 |
| | MAGEC1/CT7.1 |
| | PAGE-5/CT16.1 |
| | SSX-2/CT5.2a |
| | MAGEA12/CT1.12 |
| | BAGE/CT2.1 |
| | CT17/LIP1 |
| | SSX-4/CT5.4 |
| | SOX-2 |
| | OY-TES-1/CT23 |
| | AKAP3 |
| Differentiation | Melan-A |
| Antigens | Tyrosinase |
| | gp100 |
| Potential | IntegrinB1 = CD29 (*two variants) |
| Melanoma | CXCR4 = CD184 (* two variants) |
| Stem-like | CD166 |
| genes | Nestin |
| | Notch1 |
| | Beta-catenin |
| | MITF (*6 transcript variants) |
| | SOX9 |
| | SOX10 |
| | BRN2/POU3F2/Oct-3 |
| | Endothelin Receptor B |
| | c-kit CD117 |
| Generic | Bmi-1 |
| Stemness | nanog |
| | Shh |
| | Oct-4 |

TABLE 3

| | | Forward-Primer [Left], | Back Primer [Right]; |
|---|---|---|---|
| Name | Accession # | (SEQ ID NO) | (SEQ ID NO) |
| ABCB5 | NM_178559 | ccaaaattttattgttggatga | cagggcatgctgaaccac |
| | | (1) | (2) |
| ABCG2 | NM_004827 | tggcttagactcaagcacagc | tcgtccctgcttagacatcc |
| | | (3) | (4) |
| BAGE1/CT2.1 | NM_001187 | ggcctgagcggtaggagt | ggcagacaatgccagaaaa |
| | | (5) | (6) |
| CD133/Prominin-1 | NM_006017 | gggagaacaataataggatattttgaa | cgatgccactttctcactgat |
| | | (7) | (8) |
| CD166 | NM_001627 | ggcagtggaagcgtcataa | cattctcttcaggggaaatga |
| | | (9) | (10) |
| CT10/MAGEE1 | NM_020932 | gcaggaccatgtctctggtaa | gtgcgcagtagcctttgc |
| | | (11) | (12) |
| GAGE1/CT4.1 | NM_001468 | aggtgaaaacgcctgaagaa | tgttcattaaaagccagagaatcc |
| | | (13) | (14) |
| gp100 | NM_006928 | gcaggttatctgggtcaacaa | gggatacactggctgtcctc |
| | | (15) | (16) |
| HAGE/CT13/DDX43 | NM_018665 | ttcatataaagggcttcggagt | ccgggagttgcaattatga |
| | | (17) | (18) |
| HOM-TES-85/CT28/LUZP4 | NM_016383 | cctttaaatgggcagccttt | gcttgggcctcataattgtc |
| | | (19) | (20) |
| HORMAD1/CT46 | NM_032132 | tccaaagaaaatcggaagagaa | gaagaatcaaagtgatggaggac |
| | | (21) | (22) |
| LAGE-1a | NM_172377 | tgtccggcaacctactgttt | tgcgtgatccacatcaaca |
| | | (23) | (24) |
| LDHC/CT32 var. #1 | NM_002301 | gcgccaagaaagcatttg | catcatcctcaattagcttctcaa |
| | | (25) | (26) |
| MAD-CT-2 | AK097414 | ccaccccatgatgtcagaa | caacaccagcaatgttccac |
| | | (27) | (28) |
| MAGEA1/CT1.1 | NM_004988 | gaggacaggattccctggag | gagctgggcaatgaagacc |
| | | (29) | (30) |
| MAGEA10/CT1.10 var. #1 | NM_001011543 | tcgcaggatctgacaagagtc | ttagaccaaggcctcacctc |
| | | (31) | (32) |
| MAGEA12/CT1.12 | NM_005367 | gattctcgccctgagcaac | ggcctgtctcctcagaacct |
| | | (33) | (34) |
| MAGEA2 var. #1 | NM_005361 | ggtcgacagatgcagtggt | ctgtccccctcagaacctc |
| | | (35) | (36) |
| MAGEA3/CT1.3 | NM_005362 | tgaggaggcaaggttctgag | gggcaatggagacccact |
| | | (37) | (38) |
| MAGEA4 var. #1 | NM_001011548 | gaggagcaccaaggagaaga | cagcaggcaagagtgcag |
| | | (39) | (40) |
| MAGEA6/CT1.6 var. #1 | NM_005363 | tgaggaggcaaggttctgag | gggcaatggagacccact |
| | | (41) | (42) |
| MAGEA8 | NM_005364 | gagggcctggttctgagg | gcctgttctctgcgaacct |
| | | (43) | (44) |
| MAGEA9 | NM_005365 | gagaggcctccttctgagg | tctgcgacctgaggacact |
| | | (45) | (46) |
| MAGEB1/CT3.1 var. #1 | NM_002363 | cagaaaacaggaccttgatgtg | gctgcaccccactcctta |
| | | (47) | (48) |
| MAGEC1/CT7 | NM_005462 | gcggagggaggagacttatag | acacccaggtcttcaactcc |
| | | (49) | (50) |
| MAGEC2 | NM_016249 | gaaaccccggcctgtact | acacccagttcgtcaccac |
| | | (51) | (52) |
| MAGEC3 var. #1 | NM_138702 | ttgtcctctgccccacatac | gtaaacctggggaccctgata |
| | | (53) | (54) |
| Melan-A | NM_005511 | gagaaaaactgtgaacctgtggt | gactgttctgcagagagtttctcat |
| | | (55) | (56) |
| MMA1b/CT25.1a/DSCR8 var. #2 | NM_203428 | tggagctgccatttagaaca | ggctccttcatttttgctca |
| | | (57) | (58) |
| MORC1/CT33 | NM_014429 | aaagaagatatactgatggctggag | aggctctgaatgaccacctc |
| | | (59) | (60) |
| NA17 | X91652 | ggtggtggtggttgtttttc | tctaggggtatcacggggtag |
| | | (61) | (62) |
| NA88A/CT18 | NM_014468 | gccgtcagcatcaaggag | ggtatttggctccttactcaacc |
| | | (63) | (64) |
| Nestin | NM_006617 | tgcgggctactgaaaagttc | tgtaggccctgtttctcctg |
| | | (65) | (66) |
| NXF2/CT39 var. #1 | NM_017809 | cattctcattgaaaaggagttgg | tcgttttgtgaaaaatgactctg |
| | | (67) | (68) |
| NY-ESO-1 | NM_001327 | ccggcaacatactgactatcc | atcaacagggaaagctgctg |
| | | (69) | (70) |
| NYSAR35/CT37/FMR1NB | NM_152578 | ccatttattgccgctctctt | tcctgcctttgtaaatcatcg |
| | | (71) | (72) |
| PAGE-5/CT16.1 var. #1 | NM_130467 | ccagttggacctgtgattgtc | ggtggttcctcttcttgacg |
| | | (73) | (74) |
| PRAME var. #1 | NM_006115 | cgaggcttcagggtacagc | cctcagagagttcaccacacc |
| | | (75) | (76) |
| SAGE/CT14 | NM_018666 | caaccagtagctgataatgtcttgtc | ggatattgtgagcgatggtagc |
| | | (77) | (78) |
| SCP-1/SYCP-1/CT8/HOM-TES-14 | NM_003176 | ggactaaaagactctgatttggaga | ttcagcttctgtacttactttccatt |
| | | (79) | (80) |
| SGY-1/CT34/DKKL1 | NM_014419 | gcttcgagggtgatttgaag | tggccttctggatgggta |
| | | (81) | (82) |
| SPANXC/CT11.3 | NM_022661 | caacgaggtgaatgagacga | gtcgaggactcagatgtttca |
| | | (83) | (84) |
| SPO11/CT35 var. #1 | NM_012444 | gcacctgcattcacgataga | ccatctgaagacccacagaat |
| | | (85) | (86) |
| SSX-1/CT5.1 | NM_005635 | cgcaaccactgctttgtct | gtgtcgtctccgttcatgg |
| | | (87) | (88) |
| SSX-2/CT5.2a var. #1 | NM_003147 | tggattcttccaaaatcagagtc | caaccgtgggtctccttg |
| | | (89) | (90) |
| SSX-4/CT5.4 var. #1 | NM_005636 | tcaggttgaacgtcctcaga | gcttcttgggcatgatcttc |
| | | (91) | (92) |
| SSX-5 var. #1 | NM_021015 | cctaaccgtgggaatcagg | cttctcgggcgtgatcttc |
| | | (93) | (94) |
| TAF7L/CT40/(TAF2Q) | NM_024885 | gaggacactcaaacggatgc | cctggctttcactcatgtctt |
| | | (95) | (96) |
| TPX1/CT36/Crisp-2 | NM_003296 | actgcaggaaggtctgtgct | aggagggccatggtgtataa |
| | | (97) | (98) |
| Tyrosinase | NM_000372 | gctgccaatttcagctttaga | ccgctatcccagtaagtgga |
| | | (99) | (100) |
| XAGE-1a/CT12-1a var. #1 | NM_020411 | cccaaaaagaagaaccagca | tgcagatcaccttcatgtc |
| | | (101) | (102) |

### Example 3

### Characterization of the Proliferative Potential of MRCs In Vivo and In Vitro and Refining Tissue-Culture Techniques to Expand Populations of these Cells for Further Study

A number of studies described below require substantial numbers of cells in order to evaluate biological function. Consequently it is important to valuate the growth characteristics of MRC and to assess phenotypic evolution as these cells proliferate.

### Identification of Repopulating Cells

Preliminary studies have identified an initial phenotype based on a single marker. Using a panel of known stem cell markers, the study of these cells is expanded to characterise their cell surface phenotype in greater detail for the purposes of cell sorting and (potentially) further sub-classification. Progeny are studied in longitudinal experiments aimed at assessing the stability of their phenotype, maturation and senescence.

Refinement of Tissue-Culture Techniques to Expand Populations of these Cells for Further Study

CD133hi cells are rare and initial studies in standard tissue culture media have shown that asymmetrical proliferation of these cells yields a majority of cells with little capacity to divide and a minority which retains proliferative potential. Methods for expanding them without loss of clonogenicity are explored in order to determine whether it is possible to generate large numbers of MRC for more detailed studies. Culture conditions including the evaluation of tissue culture media, growth factors and other additives known to support the growth of stem cells are tested in vitro.

### In Vivo Growth Characteristics.

Murine CTAg homologs have not been identified conclusively in at least some cases, so experiments to explore the in vivo growth characteristics of the MRCs are undertaken in SCID mice. Melanoma lines are selected on the basis of: (i) known in vivo growth characteristics, where an existing literature exists (e.g., SK-MEL 28³⁹); and (ii) heterogeneity of CD133 and CTAg expression. The LAR series have not previously been assessed in vivo and preliminary experiments are performed to evaluate their suitability for these experiments. Characteristics to be assessed include local tumour growth and development of distant metastases following subcutaneous and intravenous injection. The proliferation and metastatic characteristics of sorted CD133hi cells are assessed following CFSE labeling.

### Example 4

### Evaluation of the Impact of Targeting CTAg in MRCs Using Immune Targeting

Whilst NY-ESO-1 is the initial target for these experiments, a variety of additional candidate targets will be identified as a result of the experiments described above. The experiments described below are extended to these other molecular targets depending on the findings of those studies. Furthermore the effects of targeting combinations of molecules also is assessed for additive or synergistic effects on molecules which have linked functions.

### Immune Targeting of MRCs

Having demonstrated the differential expression of CTAg in CD133⁺ melanoma stem-like cells we examined whether this difference could be exploited immunologically. The initial observation which prompted this line of research led us to believe that immune targeting of MRCs could be a highly effective clinical strategy. However, ‘stem cell’-like germ cells may potentially be ‘immune privileged’ and excluded from immune recognition. To determine whether MRCs can be targets for immune recognition, they are characterised for HLA class I and H expression as well as a variety of molecules required to generate MHC-peptide complexes on their surface. Biochemical studies of the proteasome determine which sub-units are utilised, since this can affect the repertoire of peptides generated and hence susceptibility of the cells to vaccine-induced T cells.

T cell lines against known antigens are used as probes for the recognition of Ag/MHC complexes using the intracellular cytokine staining (ICS) gamma interferon assay. T cell lines are derived from peripheral blood lymphocytes drawn from patients who have been previously studied and found to have T-cell responses against NY-ESO-1 epitopes³¹. Such patients may have either spontaneous (naturally arising) or vaccinated CD8+ and/or CD4+ responses⁴¹. Tumour recognition is assessed by ICS for gamma interferon. Cytotoxicity can be assessed in vitro by either standard cytotoxicity methods and was assessed by adapting the colony assay to demonstrate immune targeting of clonogenic cells, ‘MRC cytotoxicity’ (see methods). In this assay, melanoma cell lines which have heterogeneous CTAg expression were assessed. Initial experiments targeted NY-ESO-1.

Unsorted melanoma cell line LM-Mel-34 was selected as it has near ubiquitous Melan-A expression and sparse NY-ESO-1 expression (FIG. 11a) which is enriched in the CD133⁺ population.

A clonogenic cell cytotoxicity assay was performed. Unsorted LAR34 melanoma cells, which are HLA-A2 and HLA-Cw3 positive, were co-incubated with antigen-specific CTL prior to performing a colony forming (CFU) assay in soft agar. As shown in FIG. 11b, clonogenicity of these cells was eliminated by CTLs that recognized a NY-ESO-1 peptide (NY-ESO-192-100, LAMPFATPM; SEQ ID NO:103), but not by CTLs that recognized a Melan-A peptide (Melan-A26-35, A27Lsub: ELAGIGILTV; SEQ ID NO:104) specific CTL or an irrelevant control peptide of Epstein-Barr virus (EBV BMLF1280-288, C282Asub: GLATLVAML; SEQ ID NO:105).

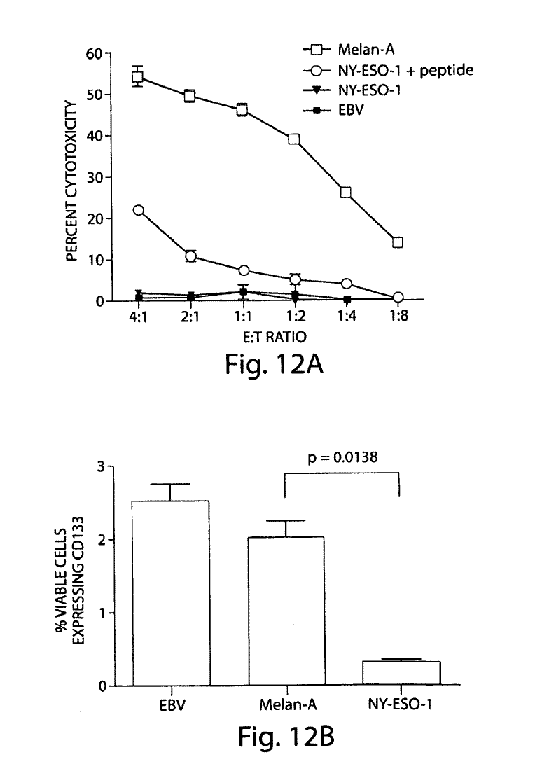

Unsorted cells were labeled and co-incubated with cytotoxic T lymphocytes (CTLs) at increasing effector-target ratios in a standard cytotoxicity assay (FIG. 12a). Melan-A CTLs showed robust killing of these cells in bulk, but there was little killing by NY-ESO-1 or irrelevant control EBV specific CTLs, unless exogenous peptide was added. In contrast, when CTLs and LM-MeI-34 melanoma cells were co-incubated under identical conditions, NY-ESO-1 but not Melan-A specific CTLs were able to specifically target the CD133⁺ cells (FIG. 12b).

An intracellular cytokine staining assay (FIG. 13) showed that LAR34 melanoma cells triggered interferon-γ production in Melan-A specific CTL, but little IFN-γ response in NY-ESO-1 or EBV BMLF1 specific CTL.

Other CTAg also are targeted and selected depending on the results of Ag expression patterns as described above and availability of antigen-specific T cells. These currently include MAGEA1, MAGEA3, MAGEA4 and MAGEA10. It is anticipated that clonogenic cells are be eliminated from unsorted cultures by targeting CTAg, even if those cell lines only express the relevant CTAg in a minority of cells. For CTAg in which the in vitro results are successful, these experiments are extended into in vivo studies. Models are established in which the eradication of clonogenic metastases following the adoptive transfer of CTAg-specific CTL is evaluated. These experiments show that CD8+ T lymphocytes specific for CTAg which are only present on a subset of melanoma cells are able to either prevent the development of metastases or eradicate melanoma by killing MRC in these tumours.

### Methods

1. Tumour acquisition & melanoma cell lines: Tumor samples were collected from patients with melanoma with their informed consent and either snap frozen in liquid nitrogen and stored at −70° C. in OCT, or formalin-fixed and paraffin-embedded. Tissue sections were cut for immunohistochemistry. The LM-Mel-melanoma cell were established in our laboratory as reported previously⁵⁰, while the cell line SK-MEL-28 was provided by the Ludwig Institute for Cancer Research, New York Branch⁵¹. Fresh tumour samples were used to derive approximately 50 melanoma cell lines (LAR: Ludwig Austin Repat.). Protocols were approved by the Human Research Ethics Committee, Austin Health, Melbourne, Australia. Cell lines were cultured in RPMI 1640 (Invitrogen, Mulgrave, Australia) supplemented with 10% fetal calf serum (CSL, Victoria, Australia), 2 mM glutamine, 25 mM HEPES, 100 U/ml penicillin, and 100 μg/ml streptomycin, and passaged with 2 mM EDTA in PBS, pH 7.4. Cell lines for self-renewal and clonogenicity experiments were passage 10 or less.

### 2. Immunohistochemistry:

Separated cells were cultured on glass slides in Flexiperm chamber wells, or cell block preparations were made (thrombin and plasma), to generate frozen (−80° C.) or fixed, paraffin-embedded sections. Specimens were sectioned and stained with αhE2 mouse anti-human prominin-1³³ at 1:500 dilution, and other antibodies as previously described⁵².

3. Cell sorting: (i) Flow cytometry. For flow cytometry passaged cells were blocked in 10% normal human serum in PBS, stained with anti-CD133 antibody (AC133-PE or 293C3-APC; Miltenyi Biotec, Bergisch Gladbach, Germany) at 1:50 dilution for 15 minutes at 4° C., washed to 20 times volume with PBS, resuspended and analyzed immediately on a FACSCalibur (Becton Dickson, San Jose, Calif.), with subsequent data analysis on FloJo (Tree Star Inc, OR). (ii) Immunomagnetic bead separation: To enrich or deplete the CD133⁺ population, passaged cells were washed in PBS, and again in MACS buffer (0.5% BSA, 2 mM EDTA in PBS pH 7.4), then labeled with either pure AC133 and rat-antimouse beads, or directly with AC133-labelled magnetic beads (Miltenyi Biotec) and separated on MACS columns according to the manufacturer's instructions. Typical purities were 90% CD133⁺ and 0.3% CD133^{neg}. To increase yield and purity CD133⁺ cells were expanded in culture for 1-2 weeks then further enriched by MACS.
4. Immunofluorescence: Cultured cells, clotted cell blocks and frozen sections were prepared as above and positioned on coverslips are blocked with 1% goat serum in PBS and incubated with primary antibodies (rabbit antihuman CD133, AbCam; mouse monoclonal antibodies to CTAg as above dilutions) overnight then stained with secondary antibodies (goat anti-rabbit Alexa Fluor 488 and goat anti-mouse Alexa Fluor 546) for one hour at 4° C. in darkness in 1% goat serum. Slides were washed three times in PBS, dehydrated with 70% ETOH, and twice with 100% EtOH, then dewaxed with xylene before mounting in DPX on slides. Application of CrystalMount (Biomeda) preceded dehydration and mounting in DePeX (BDH).
5. Quantitative RT-PCR: To quantitate the copy number of mRNA for genes of interest, qRT-PCR were performed using probes from the Universal ProbeLibrary (Roche Applied Science, Indianapolis, Ind.) with a Taqman 7700 (Applied Biosystems, Foster City, Calif.). RNA from sorted cells were prepared using a Qiagen RNeasy Mini-kit (Qiagen, Hilden, Germany). cDNA was synthesized 20-uL reaction, with Zug total RNA, 1 ug of random hexamers (Promega), 1 mmol/L deoxynucleoside triphosphates (Applied Biosystems), 40 units of RNase inhibitor (Promega), and 10 units Moloney murine leukemia virus reverse transcriptase (Invitrogen). PCR was set up with 1 uL cDNA using probes and primers designed across intron/exon boundaries using Roche website (www.roche-applied-science.com/sis/rtpcr/upl/adc.jsp) and verified using BLAT (www.genome.ucsc.edu/cgi-bin/hgBlat). Primers are exemplified in Table 3.
6. Soft Agar colony assay, limiting dilution and proliferation assays: To assess clonogenicity, cells CD133hi, CD13310 and bead-labelled but unseparated cells were cultured in RF10 in 0.3% agar (Bactoagar) on a 0.5% agar base layer and incubated at 37° C. in a humidified atmosphere with 5% CO₂. A colony is defined as >40 cells.

To assess average colony formation potential, cells were plated at 1000/well in 1 ml in 6 well plates, and colonies were counted using low magnification 14 or 28 days later.

To determine the minimum number of cells required to form a single new colony, cells were plated at limiting dilution analysis (500 to 10 cells/well) in 200 ul RPMI 10% FCS in 0.3% agar in 96 well flat-bottom plates. The number of wells without colonies at each dilution was counted 14 to 28 days later, and the natural log of this fraction was calculated at 37%.

To assess proliferation of the various cell populations, 500 cells were plated in replicates into 96-well plates and cell viability was measured by addition of MTS (Promega, Madison, Wis., USA) according to the manufacturer's instructions, and relative absorbance measured at 490 nm was calculated compared to MTS in media alone.

7. Single cell sorting: Melanoma cell lines were grown to sub-confluence, passaged and labeled with AC133-PE or 293C3-APC at 1:50 dilution and sorted into 96 well plates. The MoFlo Cyclone cell sorter (ImmunoID, Dept of Immunology and Microbiology, University of Melbourne) was calibrated prior to sorting each plate, and wells were visually inspected to ensure only one cell per well was present. Wells with proliferating cells were identified by 7 days and then inspected weekly, and discarded if no growth had occurred by three weeks.
8. T cell culture and cloning method: The culture method has been published³⁴. Briefly, frozen PBMC are used. For T cell stimulation with known minimum CD8+ T cell epitope peptides, 5×10⁶ cells are pulsed with the peptide at ˜1 μg/ml at room temperature (RT) for 60 min, then washed; for stimulating T cells with longer than minimum length peptides, 5×10⁶ PBMC are incubated with the longer peptide at ˜10 μg/ml for 60 min in minimum volume of RPMI containing 10% FCS(R-10) with 10 u/ml human recombinant IL-2. The peptide/cell mixture is then diluted in 24-well plate with R-10 containing IL-2. The T cells are cultured for 11-13 days before being used in functional assays. Cloning is performed using enriched (phycoerythrin (PE)-tetramer and anti-PE-MACS beads) or MoFlo sorted T_{CD8}+. Cloned cells are expanded under PHA stimulation with irradiated B-LCL and allo-PBMC feeder cells.
9. Intracellular cytokine staining (ICS) and tetramer staining of Ag-activated T cells. Briefly, peptide was added to cultured T cells at 10 μg/ml in R-10 (to allow serum-mediated peptide processing³⁵) along with 10 μg/ml Brefeldin A (BFA). The cells were incubated for 4 hours, harvested and stained with anti-CD4-PE and anti-CD8-Cychrome, washed and fixed with 1% paraformaldehyde. The cells were further stained with anti-IFNγ-FITC in the presence of 0.2% saponin. For tetramer staining, PE-conjugated tetramer was added to PBMC or cultured T cells at RT for 20 min, followed with addition of anti-CD8-FITC antibody for a further 20 min. At least 100,000 stained cells were acquired on a FACScalibur and analysed with Flowjo software.

### 10. Cytotoxic T-lymphocyte Preparation

Peripheral blood drawn from HLA-A2 positive consenting melanoma clinical trial patients was separated by Ficoll-density gradient centrifugation and the mononuclear cell layer cryopreserved. 5×10⁶ frozen peripheral blood leukocytes were thawed and resuspended in RF10 and pulsed with 10⁻⁸M of cognate peptide (HLA-A2 restricted Melan-A26-35 (A27Lsub: ELAGIGILTV; SEQ ID NO:104) and EBV BMLF1₁₂₈₀₋₂₈₈ (C282Asub: GLATLVAML; SEQ ID NO:105) incubated at 37° C. for 45 h, then washed thoroughly and resuspended in RF10 containing IL-2 (25 IU/ml; Cetus, Emeryville, Calif., USA). The cells were further incubated in the same media at 37° C., 5% CO₂ for 10-14 days, and passaged to maintain an approximate cell density of 2×10⁶/ml. NY-ESO-1 ESO1b₁₅₇₋₁₆₅ specific A2 restricted T-cell clones were cultured as previously described.

### 11. Cellular Cytotoxicity

LM-Mel-34 HLA-A2 restricted targets were labeled with 5 μCi/le6 targets ⁵¹Cr (Perkin Elmer, Boston, Mass.) in 0.5 ml RF10 for 2 hours at 37° C. After two washes, labeled cells were resuspended in 1 ml RPMI 1640 medium, and controls were pulsed with 1 μg/ml of cognate peptide, and incubated at 37° C. for another 30 min. After two washes, the targets were co-cultured with effector cells in 96-well, round-bottomed microtitre plates at stated effector-to-target (E:T) ratios for 4 h. At the end of the incubation period, 50 μlof supernatants were counted on a Cobra II Auto Gamma scintillation counter (Packard Instruments, Downers Grove, Ill.). The results were expressed as the percentage of specific cytotoxicity, calculated as 100×[test-spontaneous ⁵¹Cr release (%)]/[maximum-spontaneous ⁵¹Cr release (%)]. To assess specific cytotoxicity, CTL and targets were co-incubated under similar conditions, and viability and apoptosis were assessed on flow cytometry with 7-AAD (Sigma-Aldrich) and Annexin V (BD Biosciences, San Diego Calif.) according to the manufacturer's instructions.

12. In vivo cytotoxicity: Xenograft models are established in severe combined immunodeficient (SCID) mice using melanoma cell lines known to be heterogeneous for CD133 and CTAg expression. The conditions for establishing and optimizing this model are outlined in the Examples herein. Human effector cells are generated as above and are adoptively transferred. The eradication or control of clonogenic metastases is assessed.

### REFERENCES

1. Bonnet D, Dick J E. Human acute myeloid leukemia is organized as a hierarchy that originates from a primitive hematopoietic cell. Nat. Med. 1997; 3:730-737.
2. Hill R P. Identifying cancer stem cells in solid tumors: case not proven. Cancer Res. 2006; 66:1891-1895; discussion 1890.
3. Polyak K, Hahn W C. Roots and stems: stem cells in cancer. Nat. Med. 2006; 12:296-300.
4. Al-Hajj M, Wicha M S, Benito-Hernandez A, Morrison S J, Clarke M F. Prospective identification of tumorigenic breast cancer cells. Proc Natl Acad Sci USA. 2003; 100:3983-3988.
5. Singh S K, Clarke I D, Hide T, Dirks P B. Cancer stem cells in nervous system tumors. Oncogene. 2004; 23:7267-7273.
6. Brabletz T, Jung A, Spadema S, Hlubek F, Kirchner T. Opinion: migrating cancer stem cells—an integrated concept of malignant tumour progression. Nat Rev Cancer. 2005; 5:744-749.
7. Kim J J, Tannock I F. Repopulation of cancer cells during therapy: an important cause of treatment failure. Nat Rev Cancer. 2005; 5:516-525.
8. Hamburger A W, Salmon S E. Primary bioassay of human tumor stem cells. Science. 1977; 197:461-463.
9. Fang D, Nguyen T K, Leishear K, et al. A tumorigenic subpopulation with stem cell properties in melanomas. Cancer Res. 2005; 65:9328-9337.
10. Grichnik J M, Burch J A, Schulteis R D, et al. Melanoma, a tumor based on a mutant stem cell? J Invest Dermatol. 2006; 126:142-153.
11. Frank N Y, Margaryan A, Huang Y, et al. ABCB5-mediated doxorubicin transport and chemoresistance in human malignant melanoma. Cancer Res. 2005; 65:4320-4333.
12. Shmelkov S V, St Clair R, Lyden D, Rafii S. AC133/CD133/Prominin-1. Int J Biochem Cell Biol. 2005; 37:715-719.
13. Simpson A J, Caballero O L, Jungbluth A, Chen Y T, Old U. Cancer/testis antigens, gametogenesis and cancer. Nat Rev Cancer. 2005; 5:615-625.
14. Scanlan M J, Simpson A J, Old U. The cancer/testis genes: review, standardization, and commentary. Cancer Immun. 2004; 4:1.
15. Jungbluth A A, Chen Y T, Stockert E, et al. Immunohistochemical analysis of NY-ESO-1 antigen expression in normal and malignant human tissues. Int J. Cancer. 2001; 92:856-860.
16. Chen Y T, Boyer A D, Viars C S, Tsang S, Old L J, Arden K C. Genomic cloning and localization of CTAG, a gene encoding an autoimmunogenic cancer-testis antigen NY-ESO-1, to human chromosome Xq28. Cytogenet Cell Genet. 1997; 79:237-240.
17. Ayyoub M, Taub R N, Keohan M L, et al. The frequent expression of cancer/testis antigens provides opportunities for immunotherapeutic targeting of sarcoma. Cancer Immun. 2004; 4:7.
18. Claus R, Almstedt M, Lubbert M. Epigenetic treatment of hematopoietic malignancies: in vivo targets of demethylating agents. Semin Oncol. 2005; 32:511-520.
19. Coral S, Sigalotti L, Altomonte M, et al. 5-aza-2′-deoxycytidine-induced expression of functional cancer-testis antigens in human renal cell carcinoma: immunotherapeutic implications. Clin Cancer Res. 2002; 8:2690-2695.
20. Coral S, Sigalotti L, Colizzi F, et al. Phenotypic and functional changes of human melanoma xenografts induced by DNA hypomethylation: Immunotherapeutic implications. J Cell Physiol. 2005.
21. Gattei V, Fonsatti E, Sigalotti L, et al. Epigenetic immunomodulation of hematopoietic malignancies. Semin Oncol. 2005; 32:503-510.
22. Hong J A, Kang Y, Abdullaev Z, et al. Reciprocal binding of CTCF and BORIS to the NY-ESO-1 promoter coincides with derepression of this cancer-testis gene in lung cancer cells. Cancer Res. 2005; 65:7763-7774.
23. Lethè B, Lucas S, Michaux L, et al. LAGE-1, a new gene with tumor specificity. Int J. Cancer. 1998; 76:903-908.
24. Sigalotti L, Coral S, Altomonte M, et al. Cancer-testis antigens expression in mesothelioma: role of DNA methylation and bioimmunotherapeutic implications. Br J. Cancer. 2002; 86:979-982.
25. Utsunomiya T, Inoue H, Tanaka F, et al. Expression of cancer-testis antigen (CTA) genes in intrahepatic cholangiocarcinoma. Ann Surg Oncol. 2004; 11:934-940.
26. Weiser T S, Guo Z S, Ohnmacht G A, et al. Sequential 5-Aza-2′-deoxycytidine-Depsipeptide FR901228Treatment Induces Apoptosis Preferentially in Cancer Cells and Facilitates Their Recognition by Cytolytic T Lymphocytes Specific for NY-ESO-1. J. Immunother. 2001; 24:151-161.
27. Satie A P, Rajpert-De Meyts E, Spagnoli G C, et al. The cancer-testis gene, NY-ESO-1, is expressed in normal fetal and adult testes and in spermatocytic seminomas and testicular carcinoma in situ. Lab Invest. 2002; 82:775-780.
28. Takahashi K, Shichijo S, Noguchi M, Hirohata M, Itoh K. Identification of MAGE-1 and MAGE-4 proteins in spermatogonia and primary spermatocytes of testis. Cancer Res. 1995; 55:3478-3482.
29. Barr I G, Mitchell G F. ISCOMs (immunostimulating complexes): the first decade. Immunology And Cell Biology. 1996; 74:8-25.
30. Ennis F A, Cruz J, Jameson J, Klein M, Burt D, Thipphawong J. Augmentation of human influenza A virus-specific cytotoxic T lymphocyte memory by influenza vaccine and adjuvanted carriers (ISCOMS). Virology. 1999; 259:256-261.
31. Davis I D, Chen W, Jackson H, et al. Recombinant NY-ESO-1 protein with ISCOMATRIX adjuvant induces broad integrated antibody and CD4+ and CD8+ T cell responses in humans. Proc Natl Acad Sci USA. 2004.
32. Yu Y, Flint A, Dvorin E L, Bischoff J. AC133-2, a novel isoform of human AC133 stem cell antigen. J Biol. Chem. 2002; 277:20711-20716.
33. Florek M, Haase M, Marzesco A M, et al. Prominin-1/CD133, a neural and hematopoietic stem cell marker, is expressed in adult human differentiated cells and certain types of kidney cancer. Cell Tissue Res. 2005; 319:15-26.
34. Jackson H M, Dimopoulos N, Chen Q, et al. A robust human T-cell culture method suitable for monitoring CD8+ and CD4+ T-cell responses from cancer clinical trial samples. J Immunol Methods. 2004; 291:51-62.
35. Sherman L A, Burke T A, Biggs J A. Extracellular processing of peptide antigens that bind class I major histocompatibility molecules. J Exp Med. 1992; 175:1221-1226.
36. Houghton A N, Eisinger M, Albino A P, Cairncross J G, Old L I. Surface antigens of melanocytes and melanomas. Markers of melanocyte differentiation and melanoma subsets. J Exp Med. 1982; 156:1755-1766.
37. Zhang S C, Wernig M, Duncan I D, Brustle O, Thomson J A. In vitro differentiation of transplantable neural precursors from human embryonic stem cells. Nat. Biotechnol. 2001; 19:1129-1133.
38. Verfullie C M, Pera M F, Lansdorp P M. Stem cells: hype and reality. Hematology (Am Soc Hematol Educ Program). 2002:369-391.
39. Lee F T, Rigopoulos A, Hall C, et al. Specific localization, gamma camera imaging, and intracellular trafficking of radiolabelled chimeric anti-G(D3) ganglioside monoclonal antibody KM871 in SK-MEL-28 melanoma xenografts. Cancer Res. 2001; 61:4474-4482.
40. Shiraki T, Kondo S, Katayama S, et al. Cap analysis gene expression for high-throughput analysis of transcriptional starting point and identification of promoter usage. Proc Natl Acad Sci USA. 2003; 100:15776-15781.
41. Jackson H, Dimopoulos N, Mifsud N, et al. Striking immunodominance hierarchy of naturally-occurring CD8+ and CD4+ T cell responses to tumor antigen NY-ESO-1. The journal of immunology. 2006; in press.
42. Phan, G. Q., Yang, J. C., Sherry, R. M., Hwu, P., Topalian, S. L., Schwartzentruber, D. J., Restifo, N. P., Haworth, L. R., Seipp, C. A., Freezer, L. J., Morton, K. E., Mavroukakis, S. A., Duray, P. H., Steinberg, S. M., Allison, J. P., Davis, T. A. & Rosenberg, S. A. (2003) Proc Natl Acad Sci USA 100, 8372-7.
43. Morgan, R. A., Dudley, M. E., Wunderlich, J. R., Hughes, M. S., Yang, J. C., Sherry, R. M., Royal, R. E., Topalian, S. L., Kammula, U.S., Restifo, N. P., Zheng, Z., Nahvi, A., de Vries, C. R., Rogers-Freezer, L. J., Mavroukakis, S. A. & Rosenberg, S. A. (2006) Science.
44. Laduron, S., Deplus, R., Zhou, S., Kholmanskikh, O., Godelaine, D., De Smet, C., Hayward, S. D., Fuks, F., Boon, T. & De Plaen, E. (2004) Nucleic Acids Res 32, 4340-50.
45. Cho, H. J., Caballero, O. L., Gnjatic, S., Andrade, V. C., Colleoni, G. W., Vettore, A. L., Outtz, H. H., Fortunato, S., Altorki, N., Ferrera, C. A., Chua, R., Jungbluth, A. A., Chen, Y. T., Old, L. J. & Simpson, A. J. (2006) Cancer Immun 6, 12.
46. Reya, T., Morrison, S. J., Clarke, M. F. & Weissman, I. L. (2001) Nature 414, 105-11.
47. Dalerba, P., Cho, R. W. & Clarke, M. F. (2006) Annu Rev Med.
48. Singh, S. K., Hawkins, C., Clarke, I. D., Squire, J. A., Bayani, J., Hide, T., Henkelman, R. M., Cusimano, M. D. & Dirks, P. B. (2004) Nature 432, 396-401.
49. Belicchi, M., Pisati, F., Lopa, R., Porretti, L., Fortunato, F., Sironi, M., Scalamogna, M., Parati, E. A., Bresolin, N. & Torrente, Y. (2004) J Neurosci Res 77, 475-86.
50. Gibbs, P., Hutchins, A. M., Dorian, K. T., Vaughan, H. A., Davis, I. D., Silvapulle, M. & Cebon, J. S. (2000) Melanoma Res 10, 259-64.
51. Carey, T. E., Takahashi, T., Resnick, L. A., Oettgen, H. F. & Old, L. J. (1976) Proc Natl Acad Sci USA 73, 3278-82.
52. Barrow, C., Browning, J., MacGregor, D., Davis, I. D., Sturrock, S., Jungbluth, A. A. & Cebon, J. (2006) Clin Cancer Res 12, 764-71.
19. Vaughan, H. A., Svobodova, S., MacGregor, D., Sturrock, S., Jungbluth, A. A., Browning, J., Davis, I. D., Parente, P., Chen, Y.-T., Stockert, E., St. Clair, F., Old, L. J. & Cebon, J. (2004) Clin Cancer Res 10, 8396-8404.

Those skilled in the art will recognize, or be able to ascertain using no more than routine experimentation, many equivalents to the specific embodiments of the invention described herein. Such equivalents are intended to be encompassed by the following claims.

All references disclosed herein are incorporated by reference in their entirety.

We claim:

1. An isolated cell population comprising melanoma repopulating cells (MRCs) that express CD133 and at least one cancer-testis antigen (CTAg), and are clonogenic.

2. The isolated cell population of claim 1, wherein the CD133 is CD133-1 or a splice variant thereof.

3. The isolated cell population of claim 2, wherein the splice variant is CD133-2.

4. The isolated cell population of claim 1, wherein the MRCs have an increased expression of one or more cancer-testis antigens (CTAgs), in amount or type of CTAgs, relative to melanoma cells that are not MRCs.

5. The isolated cell population of claim 4, wherein the MRCs express one or more of NY-ESO-1, MAGEA3, MAGEA4, MAGEA1 and CT7.

6. The isolated cell population of claim 5, wherein the expression of NY-ESO-1, MAGEA3, MAGEA4, MAGEA1, and/or CT7 is higher in the MRCs than in melanoma cells (of the same origin) that are not MRCs.

7-8. (canceled)

9. The isolated cell population of claim 1, wherein the MRCs also express one or more neural lineage stem-cell markers.

10. (canceled)

11. The isolated cell population of claim 1, wherein the MRCs also express one or more markers for stem cells.

12-23. (canceled)

24. A method for treating cancer comprising
administering to a subject an effective amount of an agent or combination of agents selectively targeted to MRCs of the population of melanoma cells, wherein the agent or combination of agents kills the MRCs or inhibits the proliferation of MRCs.

25. (canceled)

26. The method of claim 24, wherein the agent is an antibody or antigen-binding fragment thereof that binds CD133 and/or a cancer-testis antigen (CTAg).

27-30. (canceled)

31. The method of claim 24, wherein the agent reduces expression of one or more CTAgs.

32-33. (canceled)

34. The method of claim 24, wherein the combination of agents comprises an antibody or antigen-binding fragment thereof that binds CD133 and one or more small interfering RNA molecules (siRNA) or other nucleic acid molecules that reduce expression of the one or more CTAgs by RNA interference.

35. (canceled)

36. A method for killing or inhibiting the proliferation of melanoma repopulating cells (MRCs), comprising the step of:
contacting a population of melanoma cells with an agent or combination of agents selectively targeted to MRCs of the population of melanoma cells, wherein the agent or combination of agents kills the MRCs or inhibits the proliferation of MRCs.

37. (canceled)

38. The method of claim 36, wherein the agent is an antibody or antigen-binding fragment thereof that binds CD133 and/or a cancer-testis antigen (CTAg).

39-42. (canceled)

43. The method of claim 36, wherein the agent reduces expression of one or more CTAgs.

44-45. (canceled)

46. The method of claim 36, wherein the combination of agents comprises an antibody or antigen-binding fragment thereof that binds CD133 and one or more small interfering RNA molecules (siRNA) or other nucleic acid molecules that reduce expression of the one or more CTAgs by RNA interference.

47. (canceled)

48. A method for identifying the presence of melanoma repopulating cells (MRCs) in an animal, comprising
administering to the animal a detectably labeled agent that binds to CD133 and/or a cancer-testis antigen (CTAg).

49. The method of claim 48, wherein the agent is an antibody or antigen-binding fragment thereof that binds CD133 and/or a CTAg.

50-52. (canceled)

53. A method for identifying the presence of melanoma repopulating cells (MRCs) in a cell sample or tissue sample, comprising
contacting the cell sample or tissue sample with a detectably labeled molecule that binds to CD133 and/or a cancer-testis antigen (CTAg).

54. The method of claim 53, wherein the agent is an antibody or antigen-binding fragment thereof that binds CD133 and/or a CTAg.

55-58. (canceled)

59. A method for isolating melanoma repopulating cells (MRCs) comprising
contacting a population of melanoma cells with one or more labeled antibodies that bind to CD133 and/or a cancer-testis antigen (CTAg) and isolating the MRCs based on the binding of the labeled antibodies to the MRCs.

60-65. (canceled)

66. The method of claim 24, wherein the agent is a combination of cytotoxic T lymphocytes (CTLs) that recognize CD133 and cytotoxic T lymphocytes (CTLs) that recognize at least one cancer-testis antigen (CTAg).

67. The method of claim 66, wherein the at least one CTAg is one or more of NY-ESO-1, MAGEA3, MAGEA4, MAGEA1 and CT7.

68. The method of claim 36, wherein the agent is a combination of cytotoxic T lymphocytes (CTLs) that recognize CD133 and cytotoxic T lymphocytes (CTLs) that recognize at least one cancer-testis antigen (CTAg).

69. The method of claim 68, wherein the at least one CTAg is one or more of NY-ESO-1, MAGEA3, MAGEA4, MAGEA1 and CT7.
